(19) United States
(12) Patent Application Publication
(10) Pub. No.: US 2008/0206219 A1
(43) Pub. Date: Aug. 28, 2008

(54) Novel Indications for Transforming Growth Factor-Beta Regulators

(75) Inventors: Lisa M. Coussens, Corte Madera, CA (US); Zena Werb, San Francisco, CA (US)

Correspondence Address: FENWICK & WEST LLP, SILICON VALLEY CENTER, 801 CALIFORNIA STREET, MOUNTAIN VIEW, CA, 94041, US

(73) Assignee: THE REGENCTS OF THE UNIVERSITY OF CALIFORNIA, Oakland, CA (US)

(21) Appl. No.: 10/567,873

(22) PCT Filed: Aug. 9, 2004
(86) PCT No.: PCT/US2004/025902

§ 371 Date: May 15, 2008

Related U.S. Application Data
(60) Provisional application No. 60/493,643, filed on Aug. 8, 2003.

(51) Int. Cl. A61K 38/44; A61K 48/00; A61K 39/395; A61K 31/138; A61P 19/02; A61P 17/06; A61P 35/00; A61K 31/616; A61K 38/45

(52) U.S. Cl. 424/94.4; 514/44; 424/141.1; 424/133.1; 514/648; 514/165; 424/94.5

(57) ABSTRACT

Methods, compounds and compositions for the modulation of TGF-β are disclosed wherein the vascular permeability in a subject is altered. The compounds can be antagonists or agonists, and can be oligonucleotides, antisense oligonucleotides, small molecules, antibodies, and the like. Compounds that modulate TGF-β, regulate TGF-β bioavailabililty, or the configuration and context of type 1 collagen can be used for the treatment or prevention of diseases caused by vascular permeability.

### CROSS-REFERENCE TO RELATED APPLICATION

This application claims the benefit of priority under 35 U.S.C. § 119(e) to U.S. provisional patent application No. 60/493,643, filed on Aug. 8, 2003, entitled “Novel Indications For Transforming Growth Factor-Beta Regulators” having inventors Lisa M. Coussens and Zena Werb, which is hereby incorporated by reference.

### GOVERNMENT INTEREST

This invention was made with support of government grants P01 CA 72006 and NIH NCI R01 CA98075 from the National Cancer Institute and National Institutes of Health. Therefore, the United States government may have certain rights in the invention.

### FIELD OF INVENTION

The present invention relates novel indications for modulators of transforming growth factor-β, and generally to compositions and methods for the prevention and treatment of conditions associated with vascular permeability.

### BACKGROUND

Transforming growth factor-β (TGF-β) is a cytokine that exists in at least three isoforms in mammals: TGF-β1, -2 and -3. At the cellular level, TGF-β response is mediated by or regulated by a variety of receptors and binding proteins, including the type I and type II receptors, which are serine/threonine kinases, β-glycan, and endoglin. TGF-β activity is also regulated by processes that alter delivery of the active cytokine to the cell surface. For example, TGF-β is secreted as a large latent complex that includes the propeptide, latency associated peptide (LAP), and a second gene product, latent TGF-β-binding protein (LTBP). Latent TGF-β is thought not to be biologically active. Conversion of the latent TGF-β into the active 25-kDa homodimer requires dissociation of LAP and LTBP in reactions, which may be mediated by proteinases, thrombospondin, plasmin, the mannose 6-phosphate/insulin-like growth factor-II receptor and acidic microenviroments. This active form of TGF-β is capable of binding to the TGF-β receptors. In another form, the 25 kD TGF-β dimer is found associated with matrix components or other plasma proteins. TGF-β that is associated with matrix components or other plasma proteins is termed mature TGF-β. This association also prevents the binding of TGF-β to the TGF-β receptors, and this form of mature TGF-β is thought not to be biologically active.

TGF-β regulates biological processes such as cell proliferation, differentiation and immune reaction. TGF-β has been found to have many actions in tissue repair, and it stimulates the synthesis of matrix proteins including fibronectin, collagens and proteoglycans. It also blocks the degradation of matrix by inhibiting protease secretion and by inducing the expression of protease inhibitors. It also facilitates cell-matrix adhesion and cell-matrix deposition via modulation of expression of integrin matrix receptors, and TGF-β upregulates its own expression. However, TGF-β has not yet been disclosed to modulate vascular permeability.

Alteration of vascular permeability is thought to play a role in both normal and pathological and physiological processes. For example, an increase in vascular permeability is associated with the generation of new blood vessels (angiogenesis). Angiogenesis is a complex process involving the breakdown of extracellular matrix (ECM), with proliferation and migration of endothelial and smooth muscle cells ultimately resulting in the formation and organization of new blood vessels (Folkman and Klagsbrun (1987) Science 235:442-7). Angiogenesis typically occurs via one of three mechanisms: (1) neovascularization, where endothelial cells migrate out of pre-existing vessels beginning the formation of the new vessels; (2) vasculogenesis, where the vessels arise from precursor cells de novo; or (3) vascular expansion, where existing small vessels enlarge in diameter to form larger vessels (Blood and Zetter (1990)) Biochem. Biophys. Acta. 1032:89-118).

Normal angiogenesis is an important process in neonatal growth, hair follicle cycling, in the female reproductive system during the corpus luteum growth cycle and in wound healing. Pathological angiogenesis has been associated with a large number of clinical diseases including tissue inflammation, asthma, diabetic retinopathy, psoriasis, cancer, arthritis, atheroma, Kaposi's sarcoma and haemangioma (Folkman (1995) Nature Medicine 1: 27-31). Thus, there is a need for methods and compositions for the modulation and/or alteration of vascular permeability.

### SUMMARY

The present invention provides methods, compounds and compositions for the modulation of vascular permeability in a subject. Vascular permeability can be decreased for the treatment or prevention of diseases in need thereof, or it can be increased for the treatment or prevention of diseases in need thereof.

In one aspect, the invention provides methods for the modulation of the levels of TGF-β to modulate vascular permeability. The modulator can be an antagonist, such as an oligonucleotide or a small molecule; it can be an antisense oligonucleotide; or it can be an antibody, such as a monoclonal antibody. The modulator can be an agonist, such as an oligonucleotide or a small molecule such as tamoxifen or aspirin. In another aspect, the modulator can increase or decrease the bioavailability of TGF-β.

In another aspect, the invention provides therapeutic agents for reducing collagen synthesis or collagen crosslinking to modulate vascular permeability in a subject.

These and other aspects of the present invention will become evident upon reference to the following detailed description. In addition, various references are set forth herein which describe in more detail certain procedures or compositions, and are therefore incorporated by reference in their entirety.

### BRIEF DESCRIPTION OF DRAWINGS

FIG. 1 illustrates an impaired vascular leakage in Colα1(I)^{r/r} mice treated with mustard oil. FIG. 1A shows diminished Evan's blue leakage in control ears treated with mineral oil (left ear), control mice treated with mustard oil (right ear) versus Colα1(I)^{r/r} ears treated with mineral oil (left ear) and mustard oil (right ear). FIG. 1B shows quantitative assessment of Evan's blue leakage in control and Colα1(I)^{r/r} mice treated with mineral oil and mustard oil. (*) p=0.0002 (Fishers). FIG. 1C shows fluorescent angiography of whole mounted ears following lectin perfusion of control mice treated with mineral oil (panel a) versus mustard oil (panel b) versus Colα1(I)^{r/r} (mice treated with mineral oil (panel c) or mustard oil (panel d). FIG. 1D shows the quantitative assessment of vascular area in control and Colα1(I)^{r/r} mice following mineral oil and mustard oil treatment. (*) p<0.04 (Fishers). FIG. 1E shows the quantitative assessment of vessel diameters in control and Colα1(I)^{r/r} mice following mineral oil and mustard oil treatment. (*) p=0.0001 (Fishers).

FIG. 2 illustrates fluorescent angiography (2A and B) of representative confocal images from Colα1(I)^{+/+} and Collα1(I)^{r/r} ears treated with MO. The confocal images showing VSMC phenotype and sites of vascular leakage in ears of Collα1(I)^{+/+} (2A) and Collα1(I)^{r/r} (2B) mice following MO stimulation as revealed by fluorescein-labeled Ricinus communis agglutinin I binding.

FIG. 3 illustrates the results from the modified Miles assay showing defect spectrum. FIG. 3A shows the Miles assay with VEGF-120 (10, 20, 40 ng), VEGF-164 (1, 5, 10 ng), and Serotonin (1, 2, 3 μg). (*) p<0.05 (Fishers). FIG. 3B shows the VEGFR2 phosphorylation is not impaired in Colα1l(I)^{r/r} mice. IP-western analysis

FIG. 4 illustrates the impaired stimulant-induced interendothelial opening in Colα1(I)^{r/r} mice in (A) lectin/ricin control mice with mineral oil (panel a); lectin/ricin control mice with mustard oil (MO; panel b), lectin/ricin Colα1(I)^{r/r} mic with mineral oil (panel c). In B, Ricin & αSMA IHC on MO-treated control mice (panels a-c). C. Low power EM of control mice skin with mineral oil (panel a) low power EM of control mice skin with mustard oil (panel b) low power EM of Colα1(I)^{r/r} mouse skin with mustard oil (panel c) high power EM of control mice skin with mineral oil (panel d) high power EM of control mice skin with mustard oil and (panel e) high power EM of Colα1(I)^{r/r} mouse skin with mustard oil (panel f)

FIG. 5 illustrates the effect of GM6001 on control versus Colα1(I)^{r/r} mice +/− mustard oil. 1.8× fold increase, =54%, (*) p<0.03, Fishers.

FIG. 6 shows Colα1(1)^{r/r} mice have increased MMP2 mRNA and activity. FIG. 6A shows the results of the gelatin zymogram on tissue lysates from control and Colα1(I)^{r/r} mice. FIG. 6B shows the FITC-gelatin substrate assay on lysates from control and Colα1(I)^{r/r} mice, +/− mustard oil, +/− 1,10 phenanthroline. FIG. 6C shows MMP2, MMP14, TIMP-2, 18S Northern blots.

FIG. 7 illustrates the MP-mediated activation of TGFβ and regulation of acute vascular response. In 7A illustrates the results from the treatment of Collα1(I)^{+/+} and Collα1(I)^{r/r} mice for 6-days with GM6001 versus vehicle renders Collα1(I)^{+/+} mice hyper-sensitive to vascular leakage induced by mustard oil (black bars) as compared to mineral oil (vehicle; white bars) and restores acute vascular responses in Collα1(I)^{r/r} mice to wild-type levels. (*) p=0.0055 (Mann-Whitney, two-tailed) vehicle-treated Collα1(I)^{+/+} mineral oil versus mustard oil; (**) p=0.0044 (Mann-Whitney, two-=tailed) GM6001-treated Collα1(I)^{+/+} mineral oil versus mustard oil; (***) p=0.0091 (Mann-Whitney, two-tailed) vehicle-treated Collα1(I)^{r/r} mineral oil versus mustard oil; (****) p 0.0263 (Mann-Whitney, two-tailed) GM6001-treated Collα1(I)^{+/+} mineral oil versus mustard oil. 7B illustrates the presence of low molecular weight ˜25 kDA reactive band correlating to mature bioavailable form of dimeric TGFBβ₁ in tissue lysates from Collα1(I)^{+/+} and Collα1(I)^{r/r} mice is reduced by treatment with GM6001. The band labeled (C) is the immunecomplexes in buffer control (no tissue lysate). Presence of murine heavy (HC) and light (LC) immunoglobiulin chains is also shown. Molecular mass standards are given in kDa on the left

FIG. 8A shows the TGF-β bioassay results on control and Colα1(I)^{r/r} tissue lysates. FIG. 8B illustrates TGFβ1 MRNA in ear skin from Collα1(I)^{+/+} (+/+) and Collα1(I)^{r/r} (r/r) mice as assessed by northern blot analysis of total RNA. 18S RNA is shown as a control (bottom panel). FIG. 8C illustrates Western blot analysis of Collα1(I)^{+/+} (+/+) and Collα1(I)^{r/r} (r/r) tissue lysates under reducing conditions using an antibody to LAP. ˜75 kDA reactive band corresponding to monomeric LAP was identified as compared to α-tubukin (loading control). Molecular mass standards are given in kDa on the left. FIG. 8D shows Western blot analysis of immunoprecipated proteins reveals presence of an 25 kDA reactive band correlating to the mature bioavailable form of dimeric TGFβ₁ in tissue lysates from Collα1(I)^{r/r} (r/r) mice that is not detectable in tissue lysates from Collα1(I)^{+/+} (+/+) mice. FIG. 8E Photos of Collα1(I)^{+/+} (left two panels) and Collα1(I)^{r/r} (right two panels) mice showing Evans blue leakage (blue staining) in ears of mice treated with antibodies to immunoglobulin or neutralizing antibodies to all TGFβ isoforms, following mineral oil (left ear) or mustard oil (MO; right ear) application. FIG. 8F illustrates the quantitative assessment of Evans blue leakage into interstitial tissue from Collα1(I)^{+/+} and Collα1(I)^{r/r} mice in panel E. Neutralization of TGFβ bioactivity restores appropriate acute vascular leakage responses in Collα1(I)^{r/r} mice. (*) p=0.0002 (Mann-Whitney, two-tailed) comparing MO-stimulated antiIgG-treated Collα1(I)^{+/+} versus MO-stimulated IgG-treated Collα1(I)^{r/r}; (**) p=0.046 (Mann-Whitney, two-tailed) comparing MO responses between antiIgG-versus antiTGFβ-treated Collα1(I)^{r/r} mice.

### DETAILED DESCRIPTION

### I. Definitions

Unless otherwise stated, the following terms used in this application, including the specification and claims, have the definitions given below. It must be noted that, as used in the specification and the appended claims, the singular forms “a,” “an” and “the” include plural referents unless the context clearly dictates otherwise. The practice of the present invention will employ, unless otherwise indicated, conventional methods of protein chemistry, biochemistry, recombinant DNA techniques and pharmacology, within the skill of the art. Such techniques are explained fully in the literature. See, e.g., T. E. Creighton, Proteins: Structures and Molecular Properties (W. H. Freeman and Company, 1993); A. L. Lehninger, Biochemisty (Worth Publishers, Inc., current addition); Sambrook, et al., Molecular Cloning: A Laboratory Manual (2nd Edition, 1989); Methods In Enzymology (S. Colowick and N. Kaplan eds., Academic Press, Inc.); Remington's Pharmaceutical Sciences, 18th Edition (Easton, Pa.: Mack Publishing Company, 1990); Carey and Sundberg Advanced Organic Chemistry 3^{rd} Ed. (Plenum Press) Vols A and B (1992).

All publications, patents and patent applications cited herein, whether supra or infra, are hereby incorporated by reference in their entirety.

The term “TGF-β” includes transforming growth factor-beta as well as functional equivalents, isoforms, derivatives and analogs thereof. The TGF-β isoforms are a family of multifunctional, disulfide-linked dimeric polypeptides that affect proliferation and differentiation of various cells types.

The term “modulator” means a molecule that interacts with a target. The interactions include, but are not limited to, agonist, antagonist, and the like, as defined herein.

The term “agonist” means a molecule such as a compound, a drug, an enzyme activator or a hormone that enhances the activity of another molecule or the activity of TGF-β or moieties capable of directly or indirectly activating the latent form of TGF-β to the active form there, and includes moieties capable of directly or indirectly stimulating the production of TGF-β or its latent form. Such TGF-β production stimulators may be TGF-β mRNA regulators (i.e., moieties that increase the production of TGF-β MRNA), enhancers of TGF-beta mRNA expression or the like. Plasmin, plasmin activators, matrix metalloproteinases, tamoxifen as well as analogs, derivatives or functional equivalents thereof are exemplary TGF-β activators useful in the practice of the present invention.

The term “antagonist” means a molecule such as a compound, a drug, an enzyme inhibitor, an antibody, or a hormone, that diminishes or prevents the action of another molecule or the activity of TGF-β, and includes moieties capable of directly or indirectly inhibiting the production of TGF-β or the latent form of TGF-β.

“Homology” refers to the percent similarity between two polynucleotide or two polypeptide moieties. Two DNA, or two polypeptide sequences are “substantially homologous” to each other when the sequences exhibit at least about 50%, preferably at least about 75%, more preferably at least about 80%-85%, preferably at least about 90%, and most preferably at least about 95%-98% sequence similarity over a defined length of the molecules. As used herein, substantially homologous also refers to sequences showing complete identity to the specified DNA or polypeptide sequence.

In general, “identity” refers to an exact nucleotide-to-nucleotide or amino acid-to-amino acid correspondence of two polynucleotides or polypeptide sequences, respectively. Percent identity can be determined by a direct comparison of the sequence information between two molecules by aligning the sequences, counting the exact number of matches between the two aligned sequences, dividing by the length of the shorter sequence, and multiplying the result by 100.

Readily available computer programs can be used to aid in the analysis of homology and identity, such as ALIGN, Dayhoff, M. O. in Atlas of Protein Sequence and Structure M. O. Dayhoff ed., 5 Suppl. 3:353-358, National Biomedical Research Foundation, Washington, DC, which adapts the local homology algorithm of Smith and Waterman Advances in Appl. Math. 2:482-489, 1981 for peptide analysis. Programs for determining nucleotide sequence homology are available in the Wisconsin Sequence Analysis Package, Version 8 (available from Genetics Computer Group, Madison, Wis.) for example, the BESTFIT, FASTA and GAP programs, which also rely on the Smith and Waterman algorithm. These programs are readily utilized with the default parameters recommended by the manufacturer and described in the Wisconsin Sequence Analysis Package referred to above. For example, percent homology of a particular nucleotide sequence to a reference sequence can be determined using the homology algorithm of Smith and Waterman with a default scoring table and a gap penalty of six nucleotide positions.

Another method of establishing percent homology in the context of the present invention is to use the MPSRCH package of programs copyrighted by the University of Edinburgh, developed by John F. Collins and Shane S. Sturrok, and distributed by IntelliGenetics, Inc. (Mountain View, Calif.). From this suite of packages the Smith-Waterman algorithm can be employed where default parameters are used for the scoring table (for example, gap open penalty of 12, gap extension penalty of one, and a gap of six). From the data generated the “Match” value reflects “sequence homology.” Other suitable programs for calculating the percent identity or similarity between sequences are generally known in the art, for example, another alignment program is BLAST, used with default parameters. For example, BLASTN and BLASTP can be used using the following default parameters: genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+Swiss protein+Spupdate+PIR. Details of these programs can be found at the following internet address:

http://www.ncbi.nlm.gov/cgi-bin/BLAST.

Alternatively, homology can be determined by hybridization of polynucleotides under conditions which form stable duplexes between homologous regions, followed by digestion with single-stranded-specific nuclease(s), and size determination of the digested fragments. DNA sequences that are substantially homologous can be identified in a Southern hybridization experiment under, for example, stringent conditions, as defined for that particular system. Defining appropriate hybridization conditions is within the skill of the art. See, e.g., Sambrook et al., supra.

The term “pharmaceutically acceptable salt” of a compound means a salt that is pharmaceutically acceptable and that possesses the desired pharmacological activity of the parent compound. Such salts, for example, include:

(1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, 2-naphthalenesulfonic acid, 4-methylbicyclo-[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like;

(2) salts formed when an acidic proton present in the parent compound either is replaced by a metal ion, e.g., an alkali metal ion, an alkaline earth ion, or an aluminum ion; or coordinates with an organic base. Acceptable organic bases include ethanolamine, diethanolamine, triethanolamine, tromethamine, N-methylglucamine, and the like. Acceptable inorganic bases include aluminum hydroxide, calcium hydroxide, potassium hydroxide, sodium carbonate, sodium hydroxide, and the like. It should be understood that a reference to a pharmaceutically acceptable salt includes the solvent addition forms or crystal forms thereof, particularly solvates or polymorphs. Solvates contain either stoichiometric or non-stoichiometric amounts of a solvent, and are often formed during the process of crystallization. Hydrates are formed when the solvent is water, or alcoholates are formed when the solvent is alcohol. Polymorphs include the different crystal packing arrangements of the same elemental composition of a compound. Polymorphs usually have different X-ray diffraction patterns, infrared spectra, melting points, density, hardness, crystal shape, optical and electrical properties, stability, and solubility. Various factors such as the recrystallization solvent, rate of crystallization, and storage temperature may cause a single crystal form to dominate.

The terms “effective amount” or “pharmaceutically effective amount” refer to a nontoxic but sufficient amount of the agent to provide the desired biological result. That result can be reduction and/or alleviation of the signs, symptoms, or causes of a disease, or any other desired alteration of a biological system. For example, an “effective amount” for therapeutic uses is the amount of the composition comprising a drug disclosed herein required to provide a clinically significant modulation in the symptoms associated with vascular permeability. An appropriate “effective amount” in any individual case may be determined by one of ordinary skill in the art using routine experimentation.

As used herein, the terms “treat” or “treatment” are used interchangeably and are meant to indicate a postponement of development of a disease associated with vascular permeability and/or a reduction in the severity of such symptoms that will or are expected to develop. The terms further include ameliorating existing symptoms, preventing additional symptoms, and ameliorating or preventing the underlying metabolic causes of symptoms.

By “pharmaceutically acceptable” or “pharmacologically acceptable” is meant a material which is not biologically or otherwise undesirable, i.e., the material may be administered to an individual without causing any undesirable biological effects or interacting in a deleterious manner with any of the components of the composition in which it is contained.

By “physiological pH” or a “pH in the physiological range” is meant a pH in the range of approximately 7.0 to 8.0 inclusive, more typically in the range of approximately 7.2 to 7.6 inclusive.

As used herein, the term “subject” encompasses mammals and non-mammals. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish and the like. The term does not denote a particular age or gender.

The compounds, composition, and methods of the present invention can be used to modulate vascular permeability. In this context, inhibition and reduction of vascular permeability refers to a lower level of measured activity relative to a control experiment in which the enzyme, cell, or subject is not treated with the test compound, whereas an increase of vascular permeability refers to a higher level of measured activity relative to a control experiment. In particular embodiments, the reduction or increase in the measured permeability is at least 10%. One of skill in the art will appreciate that reduction or increase of the measured permeability of at least 20%, 50%, 75%, 90% or 100% or any integer between 10% and 100%, may be preferred for particular applications.

### II. Modes of Carrying Out the Invention

The present invention discloses methods, compounds, and compositions for the modulation of TGF-β, the production of TGF-β, and the configuration and context of type 1 collagen. The present invention is based on the discovery that TGF-β regulates vascular permeability and that the bioavailability of TGF-β is regulated by a post-translational pathway mediated by type 1 collagen molecules and proteases present in perivascular stroma. The invention thus finds value in the treatment or prevention of disease states associated with angiogenesis and/or increased vascular permeability such as cancer, diabetes, psoriasis, rheumatoid arthritis, Kaposi's sarcoma, haemangioma, acute and chronic nephropathies, atheroma, arterial restenosis, autoimmune diseases, fibrotic disorders (Scleroderma), acute inflammation and ocular diseases with retinal vessel proliferation, such as macular degeneration.

TGF-β is released by platelets, macrophages and vascular smooth muscle cells (VSMC) at sites of vascular injury. Since VSMC and endothelial cells at the site of vascular injury can synthesize and release t-PA, a local mechanism for activating secreted TGF-β exists. The level of t-PA activity depends on expression of plasminogen activator inhibitor-1 (PAI-1), which is also synthesized in the vessel wall, and maybe up-regulated by TGF-β. In addition, TGF-β binds with high affinity to α2-macroglobulin thereby rendering TGF-β unable to bind to cell surface receptors for TGF-β. Polyanionic glycosamninoglycans, such as heparin, are also normally present in the vessel wall, and these moieties can reverse the association of TGF-β with α2-macroglobulin. The phenotypic state of the VSMC may affect the VSMC response to activated TGF-β. The phenotypic state of the VSMC may be influenced by their extracellular environment. Accordingly, the biological effects of TGF-β are subject to a variety of regulatory mechanisms. Described below are methods for modulating TGF-β.

A. Antagonists

In one aspect of the invention, the subject in need of treatment is administered one or more TGF-β antagonist. For example, the antagonist can be a small molecule, an oligonucleotide, or an antibody. A small molecule can be selected from the group consisting of SB-431542 (GlaxoSmithKline), NPC-30345 (Scios), and LY-364947 Lily Research). Further, an antagonist for TGF-β or for decreasing the production of TGF-β can be plasmin derived from plasminogen through activation by, for example, tPA (tissue plasminogen activator). Plasminogen and, therefore, plasmin activity is inhibited by lipoprotein Lp(a) or apolipoprotein(a), thereby decreasing the activation of the latent form of TGF-β.

As used herein, “antibody” includes a full sized antibody molecule or a fragment such as Fab, F(ab′)₂, Fv Fd and dAb fragments that retain specific binding of the immunogen, such as TGF-β, or its receptors. Fab fragment consisting of the VL, VH, Cl and CH1 domains; the Fd fragment consisting of the VH and CH1 domains; the Fv fragment consisting of the VL and VH domains of a single arm of an antibody; the dAb fragment consists of a VH domain. Single chain Fv fragments and a bivalent fragment including two Fab fragments linked by a disulphide bridge at the hinge region are also included. Naturally occurring antibodies as well as non-naturally occurring antibodies and fragments of antibodies that retain binding activity are also an antibody that can be used in the practice of the invention. Such non-naturally occurring antibodies can be constructed using solid phase peptide synthesis, or can be obtained, for example, by screening combinatorial libraries consisting of variable heavy chains and variable light chains.

A monoclonal antibody specific for TGF-β or its receptors that neutralizes the activity or biological effect of TGF-β can be prepared from an immunized rodent or other animal using well known methods of hybridoma development as described, for example, by Harlow and Lane, Antibodies: A laboratory manual (Cold Spring Harbor Laboratory Press, 1988). TGF-β (or its receptors) or a portion thereof can be used as an immunogen, which can be prepared from natural sources or produced recombinantly or can be chemically synthesized. Methods to identify hybridomas that produce monoclonal antibodies that function as a TGF-β specific inhibitory agent can utilize, for example, assays that detect inhibitors of binding between radiolabeled TGF-β and targets such as HepG2 cells or purified decorin.

The cDNA sequences encoding the light and heavy chains of a monoclonal antibody specific for TGF-β or its receptors can be obtained by cloning such sequences from hybridoma cells that secrete the antibody. Methods for cloning antibody genes are well known in the art. Humanized antibodies that inhibit the activity of TGF-β can be produced by grafting the nucleotide sequences encoding the complementarity determining regions (CDRs) from the rodent or other animal antibodies specific for TGF-β to nucleotide framework sequences derived from the light and heavy chain variable regions of a human immunoglobulin molecule. Human immunoglobulin variable region framework and constant region nucleotide sequences are well known in the art. A cDNA encoding a human immunoglobulin sequence can be obtained from publicly available gene repositories or can be cloned from human lymphoid cell lines also available from public cell repositories. Methods for humanizing antibodies by CDR grafting also are well known in the art. In addition, methods for using molecular modeling and mutagenesis approaches to maintain the original binding affinity and specificity of the rodent or other animal antibody when converted to a humanized form also are well known in the art. Thus, the antagonist can be, for example, the humanized monoclonal antibodies CAT-152 or CAT-192, both from Genzyme Corporation, or monoclonal antibodies ID11 (Genzyme Corporation) or 2G7 (Genentech).

In another aspect, the production or bioavailability of TGF-β can be inhibited thereby modulating vascular permeability. The production of TGF-β can be inhibited, for example, by use of antisense compounds. U.S. Pat. No. 5,683,988 discloses particular antisense oligodeoxynucleotides targeted to TGF-β and use of these to inhibit scarring. Dzau (WO 94/26888) discloses use of antisense sequences which inhibit the expression of cyclins and growth factors including TGF-β₁, TGF, bFGF, PDGF for inhibiting vascular cellular activity of cells associated with vascular lesion formation in mammals. A variety of methods can be used for introducing a nucleic acid encoding a TGF-β specific inhibitory agent into a cell at the site of injection in vivo. For example, the nucleic acid can be injected alone, can be encapsulated into liposomes or liposomes combined with a hemagglutinating Sendai virus, or can be encapsulated into a viral vector. In one aspect, the nucleic acid can be cloned into the pAct vector and the vector encapsulated into a liposome HVJ construct prior to injection.

Direct injection of a nucleic acid molecule alone or encapsulated, for example, in cationic liposomes also can be used for stable gene transfer of a nucleic acid encoding a TGF-β specific inhibitory agent into non-dividing or dividing cells in vivo (Ulmer et al. (1993) Science 259:1745-1748). In addition, the nucleic acid can be transferred into a variety of tissues in vivo using the particle bombardment method.

B. Agonists

In one aspect of the invention, the subject in need of treatment is administered one or more TGF-β agonist. For example, the agonist can be a small molecule, an oligonucleotide, or an antibody. The agonist can be tamoxifen, aspirin, heparin, aspirinate and its salts, including copper aspirinate itself (copper 2-acetylsalicylate or copper 2-acetoxybenzoate), salicylate salts such as copper salts of salicylates, including copper salicylate (copper 2-hydroxybenzoate) and the like. Agents which elevate TGF-levels are useful to prevent or treat diseases or conditions including cancer, Scleroderma, Marfan's syndrome, Parkinson's disease, fibrosis, Alzheimer's disease, senile dementia, osteoporosis, diseases associated with inflammation, such as rheumatoid arthritis, multiple sclerosis and lupus erythematosus, and other auto-immune disorders. Such agents also are useful to promote wound healing and to lower serum cholesterol levels.

C. Collagen Crosslinkers

In one aspect of the invention, the subject is administered a therapeutically effective amount of a collagen crosslinking agent thereby modulating vascular permeability. The crosslinking agent is preferably dispersed in a pharmaceutically acceptable carrier, such as a 5% or balanced saline solution. The crosslinking agent can be selected from a number of compounds capable of inducing crosslinking of collagen at non-toxic dosages. The crosslinking agent can be transglutaminase or a reducing sugar. Examples of suitable reducing sugars are selected from the group consisting of fructose, glucose, glycerose, threose, erythose, lyxose, xylose, arabinose, ribose, allose, altrose, mannose, fucose, gulose, idose, galactose, and talose. Further, the reducing sugar can be any suitable diose, triose, tetrose, pentose, hexose, septose, octose, nanose or decose.

In another aspect, the collagen crosslinking agent can contain a metal cation capable of inducing crosslinking of collagen. Examples of suitable crosslinking agents include sodium persulfate, sodium thiosulfate, ferrous chloride, tetrahydrate or sodium bisulfite. The metal cations are generally selected from the group consisting of sodium, potassium, magnesium, and calcium. The metal cations are typically salts of metal chlorides, bromides, iodides, phosphates, sulfates and acetates, or any other pharmaceutically acceptable salt.

In yet another aspect, the collagen crosslinking agent can be an enzyme. The enzyme can be horseradish peroxidase (HRP), soybean peroxidase (SBP) or peroxidase from Arthromyces ramosus. The enzyme solutions can contain additional agents, such as hydrogen peroxide, other peroxides, and the like.

In one aspect of the invention, the subject is administered a therapeutically effective amount of an inhibitor of collagen synthesis thereby modulating vascular permeability. Collagens are a superfamily of closely related distinct ECM proteins that play a role in maintaining the structural integrity of various tissues, such as bone, tendon, cartilage, ligaments, and vascular walls. Collagens are also involved in various developmental programs, such as cell adhesion, cell movement, homeostatis, tissue remodeling, and wound healing. The synthesis of collagen can be inhibited by a variety of methods and compositions known in the art. For example, antisense oligonucleotides and antisense gene to human type I collagen has been shown to be effective in inhibiting collagen synthesis. In addition, N-oxaloglycine, pyridine 2,4-decarboxylic acid-d(methoxyethyl)amide (HOE-077, Hoechst), colchicines, interferone gamma, nifedipine, phenytoin, and 7-bromo-6-chloro-3-[3-(hydroxy-2-piperidinyl)-2-oxopropyl]-4(3H)-quinazolinone (halofuginone) can be used to decrease collagen concentration. Preferably, halofuginone is used.

D. Protease Inhibitors

The plasminogen activator (PA) system has numerous functions, including regulation of extracellular proteolysis in a wide variety of physiological processes, such as tissue remodeling, cell migration, wound healing, and angiogenesis. Plasminogen activators (PA) are serine proteases that convert plasminogen into plasmin, a trypsin-like serine protease, that is responsible not only for the degradation of fibrin, but also contributes to the degradation and turnover of the extracellular matrix. Plasmin can be formed locally at sites of inflammation and repaired by limited proteolysis of its inactive precursor, plasminogen, which circulates in plasma and interstitial fluids. Plasminogen is activated by either urokinase-type plasminogen activator (u-PA) or tissue-type plasminogen activator (t-PA). These catalytic reactions generally take place at the plasma membrane (u-PA) or on a fibrin surface (t-PA). These activating enzymes are produced by a wide range of mesenchymal, epithelial and endoepithelial cells in response to a variety of cytokines and growth factors. Activated plasmin can degrade a wide range of substrates including extracellular matrix macromolecules (excluding collagens) and fibrin. The activities of plasmin and its activating proteinases are regulated extracellularly through a number of protease inhibitors including PAI-2 and plasminogen activator inhibitor-1 (PAI-1), and metalloproteinase inhibitors like marimastat.

In one aspect of the invention, the subject is administered a therapeutically effective amount of a protease inhibitor thereby modulating vascular permeability. The protease inhibitor can be serine protease inhibitors, a urokinase inhibitor, thiol protease inhibitors, acid protease inhibitors, and metalloproteinase inhibitors. Inhibitors of serine and thiol proteases, and of acid proteases and metalloproteases, are well known in the art, and many are commercially available, for example, from Boehringer Mannheim (Indianapolis, Ind.), Promega (Madison, Wis.), Calbiochem (La Jolla, Calif.), and Life Technologies (Rockville, Md.). Low molecular weight inhibitors of cysteine proteases have been described by Rich, Proteinase Inhibitors (Chapter 4, “Inhibitors of Cysteine Proteinases”), Elsevier Science Publishers (1986). Such inhibitors include peptide aldehydes, which form hemithioacetals with the cysteine of the protease active site. Other families of cysteine protease inhibitors include epoxysuccinyl peptides, including E-64 and its analogs (Hanada, K. et al. (1978) Agric. Biol. Chem 42: 523; Gour-Salin et al. (1993) J. Med. Chem. 36: 720), α-dicarbonyl compounds, reviewed by Mehdi, (1993) Bioorganic Chemistry, 21: 249, and N-peptidyl-O-acyl hydroxamates (Bromme et al. (1993) Biochim. Biophys. Acta, 1202: 271).

E. Treatment

As one of skill in the art will recognize, the timing of administering the dosage containing the TGF-β antagonists, agonists, collagen crosslinkers and/or protease inhibitors can vary. The compositions containing one or more of the above compounds can be administered to a subject as soon as possible after the onset of the symptoms. The administration of the compositions can be initiated within the first year of the onset of the symptoms, or preferably within the first 48 hours of the onset of the symptoms. The initial administration can be via any route practical, such as, for example, an intravenous injection, a bolus injection, infusion over 5 min. to about 5 hours, a pill, a capsule, transdermal patch, buccal delivery, and the like, or a combination thereof. The compositions are administered for a period of time sufficient to facilitate recovery. As one of skill in the art will recognize, the length of treatment can vary for each subject, and the length can be determined using the criteria described above. Typically, the compositions will be administered for at least 2 weeks, preferably about 1 month to about 1 year, and more preferably from about 1 month to about 3 months.

The vascular permeability modifying treatment described above can be applied as a sole therapy or optionally one or more other substances and/or treatments. The combination treatment can include simultaneous, sequential or separate administration of the individual components of the treatment, and can include surgery, radiotherapy or chemotherapy. Such chemotherapy may cover three main categories of therapeutic agent:

(i) other antiangiogenic agents that work by different mechanisms from those defined hereinbefore (for example linomide, angiostatin, razoxin, thalidomide, tumstatin);

(ii) cytostatic agents such as antioestrogens (for example tamoxifen, toremifene, raloxifene, droloxifene, iodoxyfene), progestogens (for example megestrol acetate), aromatase inhibitors (for example anastrozole, letrazole, vorazole, exemestane), antiprogestogens, antiandrogens (for example flutamide, nilutamide, bicalutamide, cyproterone acetate), LHRH agonists and antagonists (for example goserelin acetate, luprolide), inhibitors of testosterone 5α-dihydroreductase (for example finasteride), anti-invasion or anti-angiogenic (for example metalloproteinase inhibitors like marimastat and inhibitors of urolcinase plasminogen activator receptor function) and inhibitors of growth factor function, (such growth factors include for example EGF, platelet derived growth factor and hepatocyte growth factor such inhibitors include growth factor antibodies, growth factor receptor antibodies, tyrosine kinase inhibitors and serine/threonine kinase inhibitors); and

(iii) antiproliferative/antineoplastic drugs and combinations thereof, as used in medical oncology, such as antimetabolites (for example antifolates like methotrexate, fluoropyrimidines like 5-fluorouracil, purine and adenosine analogues, cytosine arabinoside); antitumour antibiotics (for example anthracyclines like doxorubicin, daunomycin, epirubicin and idarubicin, mitomycin-C, dactinomycin, mithramycin); platinum derivatives (for example cisplatin, carboplatin); alkylating agents (for example nitrogen mustard, melphalan, chlorambucil, busulphan, cyclophosphamide, ifosfamide, nitrosoureas, thiotepa); antimitotic agents (for example vinca alkaloids like vincristine and taxoids like taxol, taxotere); topoisomerase inhibitors (for example epipodophyllotoxins like etoposide and teniposide, amsacrine, topotecan).

As stated above the methods, compounds and compositions of the present invention are of interest for their vascular permeability and/or antiangiogenic modifying effects. Therefore, the invention is useful in a wide range of disease states including cancer, diabetes, psoriasis, rheumatoid arthritis, Kaposi's sarcoma, haemangioma, acute and chronic nephropathies, atheroma, arterial restenosis, autoimmune diseases, fibrotic disorders, acute inflammation and ocular diseases with retinal vessel proliferation. In particular, the practice of the invention can slow the growth of primary and recurrent solid tumors of, for example, the colon, breast, prostate, lungs and skin. In addition to their use in therapeutic medicine, the invention can also be useful as pharmacological tools in the development and standardization of in vitro and in vivo test systems for the evaluation of the effects of inhibitors or activators of TGF-β in laboratory animals such as cats, dogs, rabbits, monkeys, rats and mice, as part of the search for new therapeutic agents.

### EXAMPLES

Below are examples of specific embodiments for carrying out the present invention. The examples are offered for illustrative purposes only, and are not intended to limit the scope of the present invention in any way. Efforts have been made to ensure accuracy with respect to numbers used (e.g., amounts, temperatures, etc.), but some experimental error and deviation should, of course, be allowed for.

### Histology and Immunohistochemistry

Tissue samples were fixed by immersion in 10% neutral-buffered formalin, dehydrated through graded ethanol and xylenes, embedded in paraffin, cut by a Leica 2135 microtome into 5-μm-thick sections. Hematoxylin and eosin staining was performed using standard methods. Masson's trichrome staining was performed using the Accustain Trichrome Stains (Sigma, St. Louis, Mo.). For picro-sirius red staining, rehydrated sections were stained 5 min in Weigert's hematoxylin (Sigma) blued under running tap water 5 min, then stained 10 min in a picro-sirius red stain (0.1% Sirius red F3B (Sigma) in a saturated aqueous solution of picric acid (Sigma), washed twice in 0.1% acetic acid, dehydrated and mounted in Permount (Sigma). Slides were viewed and photographed under non-polarized and polarized light. Immmunodetection of alpha smooth muscle actin was performed on tissue pieces following injection of Ricinus communis lectin and cardiac perfusion. Tissue pieces were fixed in 4% paraformaldehyde for 4 hrs at 4° C., followed by several washes in 4° C. phosphate buffered saline (PBS) and permeabalization in 0.3% TritonX-100 overnight at 4° C. Tissue pieces were then incubated with an anti-smooth muscle actin mAB (Sigma, 1:500) diluted in 5% normal goat serum, 2.5% BSA, 0.3% TrionX-100 in PBS overnight at 4° C. on a rotating platform. This was followed by extensive washing in 4° C. PBS and mounting with Vectashield (Vector, Burlingame Calif.) mounting medium.

### Ultrastructural Electron Microscopy

Briefly, ear skin pieces were collected following cardiac perfusion, thinly sliced (˜1 mm thick) and placed in Karnovsky's fixative (1% para-formaldehyde, 3% glutaraldehyde, 0.1 M sodium cacodylate buffer, pH 7.4) at room temperature for 30 minutes before storage at 4° C. Fixed tissue were then rinsed in water, post-fixed in reduced OsO₄ (2% OsO₄ in 1.5% potassium ferrocyanide; Sigma Chemical), stained en bloc with uranyl acetate before dehydration in 100% ethanol, cleared in propyline oxide, and embedded in Eponate 12 (Ted Pella Co.). Thick section were cut and stained with toluidine blue, examined under light microscope to select areas for subsequent thin sectioning. Thin sections were cut on a Leica ultracut E microtome (Bannockburn), stained with uranyl acetate and Reynold's Lead to enhance contrast and examined with a Philips Tecnai 10 electron microscope (Eidhoven).

### Hydroxyproline Determination

Collagen content in ears and back skin from 6-wk and 6-mo old mice was determined as described by Woessner (1961) Arch Biochem Biophys 440-447 (1961). Briefly, mice were shaved and 10-30 mg wet weight of tissue and Trans-4-Hydroxy-L-Proline (Sigma-Aldrich) as standard were hydrolyzed over night in pyrex tubes at 110° C. in 1 ml 6N HCl. Samples were subsequently filtered through Low Binding Durapore membrane filter devices and stored at −20° C. until analysis. Aliquots were then speed-vac dried and hydroxyproline content determined as described by Woessner. For generation of the standard curve, samples of known concentrations were used in the linear range (0.45-4.5 μg) and all samples were analyzed in triplicate. For determination of collagen content, 1.0 μg hydroxyproline was used as an equivalent to 6.94 μg of collagen.

### Miles Assay

Evans blue (EB) dye (30 mg/kg in 100 μl PBS; Sigma-Aldrich) was injected into the tail vein of 7- to 8-week-old mice. In some experiments, after 1-min, 30 μl of 5% mustard oil (Phenyl Isothiocyanate, 98%, Sigma-Aldrich) diluted in mineral oil (Sigma-Aldrich) was applied to the dorsal and ventral surfaces of the ear; the application process was repeated 15 minutes later. Isoflurane anesthesized mice were photographed 30 minutes after injection of EB dye. Anesthesized mice were then cardiac perfused, ears removed, blotted dry and weighed. EB dye was extracted from ears in 1 ml of formamide overnight to 48-hrs at 60° C. and measured spectrophotometrically at 610 nm in a SpectraMax 340™ (Molecular Devices). Data are expressed as mean±SEM. Comparisons of the amounts of dye extravasation were evaluated by Mann-Whitney statistical test with p values less than 0.05 considered significant. In some experiments, 5-min prior to the infusion of EB dye, shaved 5-to 7-week old mice were injected (10 μl) intradermally with one of the following agents at the concentrations shown (VEGF₁₂₀, R&D Systems; VEGF₁₆₄, Chemicon; histamine, Calbiochem; serotonin, Sigma-Aldrich) and the appearance of a blue spot monitored for 30 minutes at which time mice were euthanized, cardiac perfused, photographed and the area of skin surrounding the site of injection excised (˜5 mm²), photographed and EB dye extracted as above.

### Vascular Perfusions and Fluorescent Angiography

Isoflurane-anesthetized mice were injected with fluorescein-labeled Lycopersicon esculentum lectin (100 μl, 2 mg/ml; Vector Laboratories, Burlingame, Calif.) or Rhodamine-labeled Ricinus communis agglutinin I (50 μl, 5 mg/ml; Vector Laboratories, Burlingame, Calif.) into the femoral vein. Two minutes after lectin injection, mice were perfused with fixative (1% paraformaldehyde plus 0.5% glutaraldehyde in phosphate-buffered saline, pH 7.4, at 37° C.) via the ascending aorta for 2-min to fix the vasculature and flush out non-adherent leucocytes. Confocal images were acquired on a Zeiss LSM 510 META NLO with an ultrafast, tunable Coherent Ti:Sa MIRA laser with Verdi pump for multi-photon excitation.

### Immunohistochemistry

Immmunodetection of α-smooth muscle actin was performed on tissue pieces following injection of Ricinus communis lectin and cardiac perfusion as decribed above. Tissue pieces were fixed in 4% paraformaldehyde for 4-hrs at 4° C. with gentle agitation in the dark followed by several washes in 4° C. PBS and permeabolization in 0.3% TritonX-100 overnight with gentle agitation at 4° C. Tissue pieces were then incubated with Cy3-labelled anti-α-smooth muscle actin mAB (Sigma-Aldrich, Clone 1A4 #C6198, 1:500) diluted in 5% normal goat serum, 2.5% BSA, 0.3% TrionX-100 in phosphate buffered saline (PBS) overnight at 4° C. on a rotating platform, followed by extensive washing in 4° C. PBS and mounting with Vectashield (Vector) mounting medium and images acquired on a Zeiss LSM 510 META NLO with an ultrafast, tunable Coherent Ti:Sa MIRA laser with Verdi pump for multi-photon excitation.

### Protein Analysis

VEGFR2: Tissue pieces (5 mm²) from animals were collected from ears or following shaving of back skin or following injection (i.d.) of 10 μl 10 ng VEGF₁₆₄ or 0.1% BSA in PBS. Tissues were pulverized in liquid N₂ followed by lysis in ice-cold buffer containing 20 mM Tris, pH 7.6, 150 mM NaCl, 1 mM EDTA, 50 mM NaF, 1% triton X-100, 0.5% Na-deoxycholate, 0.1% SDS, 2 mM Na₂VO₄, 10 μg/ml aprotinin, 1 mM phenylmethylsulfonylfluoride and centrifuged at 10,000 rpm for 30-min at 4° C. The supernatants were recentrifuged at 10,000 rpm for 30-min at 4° C. Lysates were then incubated in a slurry of heparin-Sepharose CL-6B (Pharmacia) and incubated overnight rocking at 4C, centrifugation and equilibrated to 150 mM NaCl. Protein was dialyzed against PBS and quantified using the BioRad protein assay system (BioRad). Before immunoprecipitation, BSA was added to the pre-cleared lysates to 0.5%. Equal amounts of protein (1 mg) from lysates were used for immunopreciptations and Western blotting. Incubation of tissue lysate with goat anti-Flk-1 (Santa Cruz Biotechnology) followed by protein-G sepharose beads was performed for 2-hrs at 4C. Immunoprecipitates were washed three times with 20 mM Tris (ph 7.6), 150 mM NaCl, 0.1% Triton X-100 and bound proteins were eluted by boiling in 1×SDS-PAGE sample buffer for 5-min, followed by electrophoresis on 10% SDS-PAGE under reducing condition. The resolved proteins were transferred to a nitrocellulose membrane (BA-S85, Schleicher & Schuell). Anti-phoshoptyrosine PY-20 (Upstate Biotechnology) and anti-Flk-1 (Santa Cruz Biotechnology) antibodies were used on Western blots. Immunodetection was performed by incubation with specific peroxidase-conjugated secondary antibodies followed by enhanced chemiluminescence (ECL, Amersham).

TGFβ ELISA: Protein lysate for IP-Western and ELISA analyses were prepared from shaved back skin pieces (˜5 mm²) from 5-8 week old mice. Tissues were pulverized in liquid N₂ and solubilized in 600-800 μl lysis buffer containing 50 mM Tris, 75 mM NaCl, 10 mM EDTA, Protease Inhibitor cocktail mix without EDTA (Roche), 0.01 mg/ml Aprotinin (Sigma-Aldrich), 0.1 mg/ml Leupeptin (Sigma-Aldrich), 10 mM PMSF (Sigma-Aldrich) using a 2 ml tissue grinder (Fisher), with sonication at 4° C. and centrifugation at 4° C. 10,000×for 30 min. Protein concentration of the supernatant was determined with the BioRad DC Protein assay reagent according to manufacturers instructions (BioRad). Aliquots were kept at −80° C. The total amount of TGF-β1 in lysates was determined by using a standard protocol for quantitative sandwich enzyme immunoassay. For ELISA analysis, monoclonal antibody specific for active TGF-β1, 2, 3 (R&D System MAB1835) was used to pre-coat maxisorb immuno plates (NUNC) over night at RT (1.0 μg/ml in PBS). Prior to incubation on coated plates, lysates (100 μg) were activated by adding 1.0 N HCl (1:25) and incubated for 1-hr at 4° C. with gentle agitation. Acidified samples were neutralized by adding 1.0 N NaOH (in the ratio 1:25) and diluted with ELISA Sample Buffer (1× PBS, 0.05% Tween-20, 1.4% fatty-acid free BSA). Samples were incubated 3-hrs at RT in pre-coated maxisorb immuno plates (NUNC), which was followed by extensive washing (1×PBS, 0.1% fatty-acid free BSA, 0.05% Tween-20) and addition of 100 μl biotinylated anti-TGF-β1 antibody (R&D System BAF240) at 200 ng/ml in PBS and incubated over night at 4° C. After washing, avidin-peroxidase conjugate (Sigma-Aldrich, 1:1000) was added for 1-hr at RT followed by a 20-min incubation at RT in the dark with OPD substrate (Sigma-Aldrich). The reaction was stopped with 1.0 M H₂SO₄ and absorbance was measured at 450 (570 nm for background corrections) on a Molecular Device Spectra Max 340. Recombinant human TGF-β1 (R&D Systems) was used as the standard. The concentration of the standard curve was in the linear range (25-1000 pg/ml), six tissues samples per genotype were analyzed and all samples were analyzed in duplicate.
TGFβ, LAP and MMP14: For immunoprecipitation of TGFβ and MMP14, 4200 μg of protein lysates were pre-cleared with protein A-agarose beads (Roche) 1 hour at 4° C., followed by centrifugation at 3,000 rpm (5-min) and incubation of the supernatant with 2.0 μg of antibody for TGF-β1, 2, 3 (R&D System MAB1835) or MMP14 (Chemicon AB8102, catalytic domain; MAB3317, hemopexin domain) for 3 hours at 4° C. in HNTG buffer (20 mM Hepes, pH 7.5, 150 mM NaCl, 0.1% TritonX-100, 10% Glycerin, 10 mM Na-pyrophosphate, 10 mM Na-F, 1 mM Na-o-vadanate, 1 mM PMSF, and 10 ug.ml aprotinin). After incubation with protein agarose G or A beads (Roche) beads for an additional hour at 4° C., lysates bound to agarose beads were washed three times with HNTG buffer and bound proteins were eluted by boiling in 1× reduced SDS_PAGE sample buffer for 5-min and centrifuged at 13,000 rpm for 10-min. Tissue lysates (20 μg for LAP) or eluted immunoprecipitated complexes were separated by electrophoresis on 10% SDS-polyacrylamine gels, and transferred to nitrocellulous membranes overnight at 4° C. Membranes were blocked, incubated with primary antibodies for 1-2 hour at room temperature, washed and further incubated with secondary antibodies (BioRad, goat anti-rabbit- or goat anti-mouse-HRP conjugate 1:2,000) or strepavidin-HRP conjugate (Sigma-Aldrich, 1:20,000) for 1-hr at room temperature. Membranes were then washed and developed by using an enhanced chemiluminescence kit (ECL, Amersham Biosciences). Biotinylated-LAP antibodies (R&D System BAF246, 1:1000), biotinylated anti-TGF-β 1 antibodies (R&D System BAF240, 1:1000) and antibodies to MMP14 (Oncogene Sciences 1M397, 1:1,000; Chemicon AB8104, 1:1000 were used for detection on membranes. For loading control in LAP western analysis, rat monoclonal antibody (AbCam YL1/2, 1:5,000) against o-tubulin and goat anti-rat-HRP (Pierce, 1:2000) antibodies were used.

### RNA Analysis

Total RNA was extracted from shaved back skin or ear pieces with TRIzol reagent™ (Invitrogen) according to the manufacturers recommendations by powdering fresh-frozen tissue samples in liquid N₂, homogenizing with a microtube pestle (USA Scientific), shearing by multiple passages through a syringe and 21-gauge needle (Becton Dickinson), followed by chloroform extraction, isopropanol precipitation and ethanol wash. Northern blot analysis was performed using standard methods with 10 μg of total cellular RNA. Probes were generated by random primed labeling of DNA isolated from plasmids using standard methodology. Northern blots were hybridized at 65° C. overnight in Church buffer (0.5 M Sodium phoshate pH 7.2, 1 mM EDTA, 7% w/vol SDS, 250 μg/ml tRNA), and subsequently washed at 62° C. in 2×SSC containing 1% SDS. Probes used for hybridization were: 335 bp fragment of mMMP2 (EMBL: M84324; position: 2053-2387 bp), 335 bp fragment of mMMP14 (EMBL: NM_{—}008608; position: 54-388 bp), 669 bp fragment of mTIMP2 (EMBL: X62622; position: 2-670 bp), 974 bp fragment of mTGFβ1 (EMBL: M13177; position: 421-1395 bp) and a 207 bp fragment for 18S RNA as loading control (EMBL: J00623; position: 13-219 bp). Hybridized filters were exposed overnight on phosphor screens and analyzed in a Phosphoimager (Molecular Dynamics, Storm 860, ImageQuant 5.2 software) and additionally exposed for 1-3 days on Kodak film (Biomax MS) with Intensifier screen at −80° C.

### Substrate Conversion Assay

Shaved back skin pieces from 5-8 week old mice were pulverized in liquid N₂ and solubilized in 500 μl buffer (0.25 M sucrose, 5 mM Tris, pH 7.5, protease Inhibitor cocktail mix without EDTA (Roche), 0.25 mg/ml Pefablock (Roche), 0.01 mg/ml Aprotinin (Sigma-Aldrich) using a 2 ml tissue grinder (Fisher) and centrifuged at 4° C. 800×g for 15-min. Supernatants were centrifuged for 1-hr at 100,000×g at 4° C. Supernatants were stored at −80° C., pellets were resuspended in 100 μl solubilization buffer, homogenized by sonication at 4° C., and stored at −80° C. Protein concentration was determined with the BioRad DC Protein assay reagent according to manufacturers instructions (BioRad). Prior to assay, lysate buffers were exchanged using Micro Bio-spin chromatography columns (Bio-gelP-6; BioRad) to 10 mM Tris pH 7.5 according to the manufacturers specifications. For assay of gelatinolytic activity in lysates, 50 μg protein from the supernatant fraction was incubated at 37° C. with 400 ng DQ-gelatin (Molecular Probe) in reaction buffer (50 mM Tris, pH 7.6, 150 mM NaCl, 5 nM CaCl₂, 0.2 mM NaAzide and 0.05% BrJ35) in a total volume of 200 μl/well (black 96-well plate, Falcon). Reactions were incubated up to 5-hr at 37° C. and fluorescence measured (excitation 485 nm, emission 530 nm) every 3-min on a Microplate Spectrofluorometer (SpectraMax Gemini EM, Molecular Devices) and quantified using SoftMax Pro 4.1 software. Values shown represent the mean +/− SEM from three tissue pieces and are representative of analyses performed in triplicate, and repeated three independent times.

### Substrate Zymography

Tissue samples (ear) from 5-8 week old mice were weighed and homogenized (1:8 weight to volume) in lysis buffer containing 50 mM Tris-HCl (pH 8.0), 150 mM NaCl, 0.1% NP-40, 0.5% deoxycholate, 0.1% SDS. Soluble and insoluble extracts were separated by centrifugation (10,000×g) and subsequently stored at −80° C. Equivalent amounts of soluble extract were analyzed by gelatin zymography on 10% SDS-polyacrylamide gels copolymerized with substrate (1 mg/ml of gelatin) in sample buffer (2% SDS, 50 mM Tris-HCl, 10% Glycerol, 0.1% Bromphenol Blue, pH 6.8). After electrophoresis, gels were washed 3 times for 30-min in 2.5% Triton X-100, 3 times for 15-min in ddH₂O, incubated overnight at 37° C. in 50 mM Tris-HCl, 10 mM CaCl₂ (pH 8.2), and then stained in 0.5% Coomassie Blue and destained in 20% methanol, 10% acetic acid. Negative staining indicates the location of active protease bands. Exposure of proenzymes within tissue extracts to SDS during gel separation procedure leads to activation without proteolytic cleavage.

### Cell-Based MMP Assay

To prepare collagen gels for culture experimentation, mouse tail collagen was purified and quantified by determination of hydroxyproline content as described above. Subsequently 8 volumes of +/+ and r/r collagen (4.4 mg/ ml) were neutralized by addition of 1 volume 10×PBS containing 0.005% phenol red and 1 volume NaOH. 50 μl of MDA-MB-231 breast carcinoma cells expressing a full length human MMP14 cDNA at 5×10⁶ cells/ml in serum-free DMEM were added to 200 μl of neutralized +/+ and r/r collagen. The collagen/cell suspensions were mixed well and then four 50 μl aliquots were added per well into a 96-well culture dish (Corning) and incubated at 37° C. for 1-hr to allow collagen polymerization. 100 μl of DMEM containing 10% fetal bovine serum was then added to cells and incubated at 37° C. for 18-hr. Collagen gels were washed with 200 μl serum-free DMEM and cells were incubated in 100 μl serum-free DMEM containing human proMMP2 since the MDA-MB-231 cells express essentially no MMP2. Conditioned media were harvested after 48-hr and collagen gels washed in 200 μl of PBS. 50 μl of non-reducing SDS-PAGE sample buffer was then added to collagen gels to extract collagen bound MMP2 and after collection brought to 200 μl total volume. Equivalent amounts of supernatants and collagen bound MMP2 extracts were analyzed by gelatin zymography that were incubated 4-hr at 37° C.

### EXAMPLE 1

### Animal Husbandry

Mice were housed under conditions conforming to University of California Regulations. Colα1(I)^{r/r} mice were derived from the colony at Massachusetts General Hospital, Boston, where the mutation was targeted to the embryonic stem cells of the J1/129 strain and then introduced to the C57BL/6 strain. Backcrosses to FVB/n (N5) were performed to create an inbred line of Colα1(I)^{r/+} mice, and a breeding colony of homozygous-mutant Colα1(I)^{r/r} mice established (UCSF). Controls were progeny of wild-type Colα1(I)^{+/+} breeding pairs, which do not possess the mutated gene but which are on the same genetic background. Presence of the mutant COL1A1 allele was assessed by PCR genotyping of tail DNA using oligonucleotide primers discriminating between the wildtype allele (5′-TGGACAACGTGGTGTGGTC-3′ (SEQ ID No: 1) and TTGAACTCAGGAATTTACCTGC (SEQ ID No: 2)) versus the mutant allele (TGGACAACGTGGTGTGGTC (SEQ ID No: 3) and TGGACAACGTGGTGCCGCG (SEQ ID No: 4)) when DNA was successively amplified for 30 cycles at 95° C. 60 seconds, 59° C. 30 seconds, and 72° C. 120 seconds, to generate 300-bp product. Art known βA-hT1 transgenic mice contain a transgene where the human β-actin promoter directs expression of a human TIMP-1 cDNA that were initially generated in the CD1 mouse strain. To minimize the effect of background strain differences, βA-hT1mice were backcrossed a minimum of six generations into the FVB/n strain. The βA-hT1 transgene was followed by PCR genotyping of tail DNA using oligonucleotide primers (TGTGGGACACCAGAAGTCAAC (SEQ ID No: 5) and CTATCTGGGACCGCAGGGACT (SEQ ID No: 6)) and DNA was successively amplified for 30 cycles at 95° C. 60 seconds, 59° C. 30 seconds, and 72° C. 120 seconds, to generate a 480-bp product corresponding to a region within the human TIMP-1 cDNA. Analyses using βA-hT1⁺ transgenic mice were compared to littermate controls lacking the βA-hT1 transgene (βA-hT1⁻). Mice carrying a targeted null mutation in the MMP-2 (Itoh (1997) Journal of Biological Chemistry 272: 22389-22392) and TIMP-1 (Alexander (1992) J Cell Biol 118: 727-739)(Soloway (1996) Oncogene 13: 2307-2314) genes were individually backcrossed into the FVB/n background for 5 generations at which time they were intercrossed and homozygous null genotypes generated and compared to heterozygous littermate controls. MMP2 homozygous null mice (FVB/n, N5) and Colα1(I)^{r/r} mice (FVB/n, N5) were intercrossed to generate Colα1(I)^{r/r}/MMP2^{-/-} mice.

Neutralization of TGFβ activity in vivo was accomplished by intraperitoneal (i.p.) injections of pan-specific TGFβ antibody R & D Systems, #AB-100; 1.0 mg/ml in sterile PBS pH 7.4) at 5.0 mg/kg body weight 120-, 96- and 24-hr prior to MO challenge. Control animals received normal rabbit IgG (R&D Systems; #AB-105-C). Five animals per cohort were injected and the experiment was repeated three times. N-[(2R)-2(hydroxyamnidocarbonylmethyl-4-methylpantanoyl]-L-tryptophan ethylamide (GM6001), a broad, class-specific metalloproteinase inhibitor (Chemicon, Temecula Calif.), was administered i.p. 100 mg/kg body weight as a 20 mg/ml slurry in 4% carboxymethylcellulose (CMC) in 0.9% PBS daily for 3-days prior to cutaneous challenge. Controls were treated with a daily injection of 4% CMC in PBS. Four animals per cohort were injected and the experiment was repeated four times. This concentration of GM6001 has been demonstrated to inhibit in vivo MP activity. For all other experiments, analyses were conducted in triplicate on cohorts containing at least three mice and p values<0.05 were considered significant.

### EXAMPLE 2

### Vascular Permeability and Vasodilation Responses in Collα1(I)^{r/r} Mice

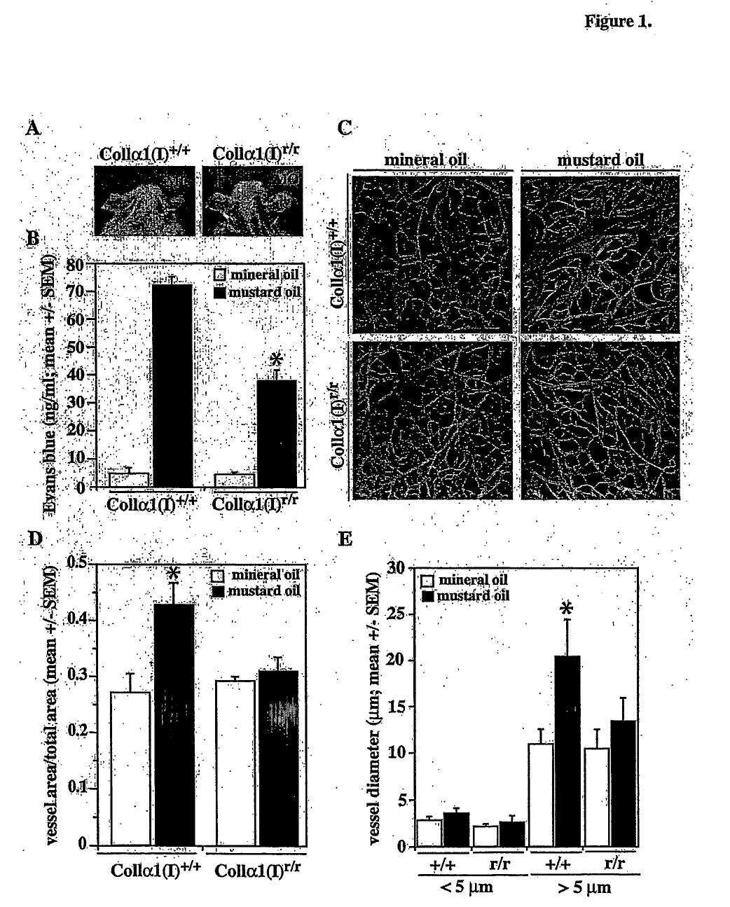

The vascular physiology in a mouse model of human Sc, e.g., Collα1(I)^{r/r} mice, was studied to determine whether an altered balance between collagen synthesis, accumulation and/or degradation was a rate-limiting factor for efficient vascular physiology prior to histopathologic appearance of Sc disease. The ears of Collα1(I)^{r/r} versus littermate control (Collα1(I)^{+/+}) mice were treated with vehicle alone (mineral oil; FIG. 1A) or mustard oil (MO; 5% in mineral oil: right ear), an inflammatory agent that induces plasma leakage, vasodilation of capillaries and inflammation in the skin (FIG. 1A). Evans blue dye (30 mg/kg in 100 μl PBS; Sigma Chemical Co., St. Louis, Mo., USA) was injected into the tail vein of the mice. After 1 minute, 5% mustard oil (Phenyl Isothiocyanate, 98%, Sigma) diluted in mineral oil (Sigma) was applied to the dorsal and ventral surfaces of the ear with a cotton swab; the application process was repeated 15 minutes later. Following MO exposure, ears of littermate control mice became moderately blue, particularly at the periphery (FIG. 1A, left panel, right ear). In contrast, ears of Collα1(I)^{r/r} mice remained pale with only a modest hint of blue (FIG. 1A, right panel, right ear). Isoflurane anesthesized mice were photographed 30 minutes after injection of Evans blue dye. Anesthesized mice were then cardiac perfused, ears removed, blotted dry, and weighed. The Evans blue dye was extracted from the ears with 1 ml of formamide overnight at 60° C. and measured spectrophotometrically at 610 nm in a SpectraMax 340™ (Molecular Devices). Data are expressed as mean±SEM. Comparisons of the amounts of dye extravasation were evaluated by the Fisher's t test with p values less than 0.05 considered significant. Organic extraction and spectrophotometric analysis of ear tissue revealed the amount of Evans blue that had ‘leaked’ out of the vasculature and into the surrounding stroma in response to MO in Collα1(I)^{r/r} mice was attenuated and ˜50% lower than in controls animals (p=0.0002, Fishers) (FIG. 1B).

Fluorescent angiography where vasculature in whole mounted tissue was visualized by confocal microscopy (FIG. 1C). Following MO treatment (27 min), animals were injected (i.v.) with fluorescein Lycopersicon esculentum lectin (100 μl, 2 mg/ml), a tomato lectin that specifically binds to the luminal surface of vascular endothelial cells. Treatment of control mice with MO (FIG. 1C, panel b) as compared to vehicle alone (FIG. 1C, panel a) resulted in a significant increase in total vascular area (FIG. 1D; p<0.04, Fishers). In contrast, vasculature in Collα1(I)^{r/r} mice was unchanged (FIG. 1C, panel d and FIG. 1D). The increase in total vessel area observed in control mice treated with MO resulted from increased vasodilation of macrovasculature (FIG. 1E; p<0.001, Fishers), a response not observed in Collα1(I)^{r/r} mice (FIG. 1E). Therefore, altered collagen metabolism in the vascular stroma of Collα1(I)^{r/r} mice rendered vascular networks less susceptible to MO-induced vascular permeability and vasodilation, resulting in diminished vascular leakage.

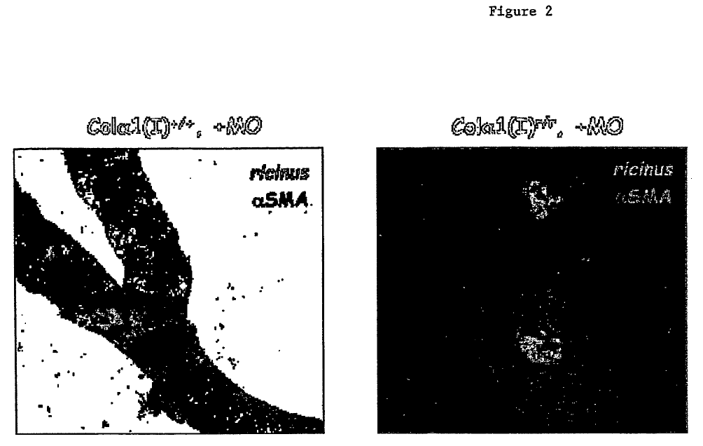

Next, it was determined whether diminished appearance of vascular leakage sites in Collα1(I)^{r/r} mice was due to altered venular coverage by vascular smooth muscle cells (VSMCs) or pericytes (PC) VSMC/PCs as compared to Collα1(I)^{+/+} mice in areas susceptible to MO-induced vascular leakage. Alpha smooth muscle actin (αSMA) is a contractile protein localized on microfilament bundles in perivascular VSMC/PCs and the location and morphology of αSMA-positive perivascular cells was determined in untreated control ears. Following MO treatment and Ricinus communis Agglutin I injection, the vasculature in Collα1(I)^{r/r} mice was found to be refractory to vasodilation and containing few sites of ricinus binding compared to Collα1(I)^{+/+} (FIG. 2). There were no discernible differences, however, between the groups of mice in either abundance, organization or morphology of αSMA-positive perivascular cells in areas where vascular leakage was evident. Therefore, the failure to mount an appropriate acute vascular response in Collα1(I)^{r/r} mice was not due to a primary defect in VSMC/PC investment, but may be related to changes in VSMC/PC function resulting in contractile failure and resistance to vascular leakage.

### EXAMPLE 3

### Vascular Responses to Mineral Oil

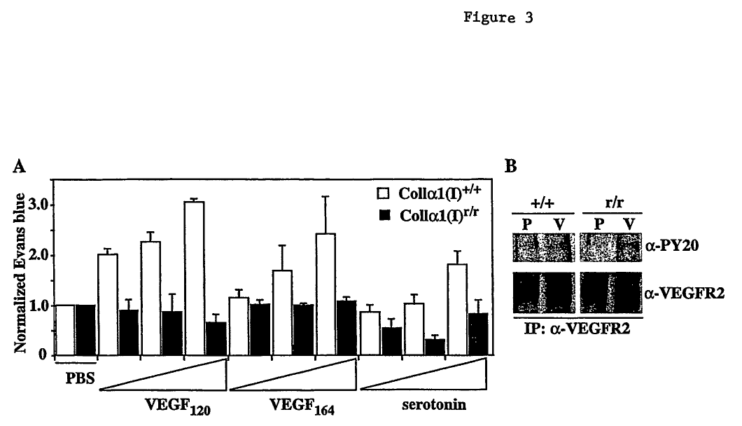

The vascular leakage in control and Collα1(I)^{r/r} mice following intradermal injection of other agents known to induce vascular leakage, e.g., VEGFA₁₂₀, VEGFA₁₆₄ and serotonin (versus vehicle alone), by interacting with distinct cell surface receptors, e.g., VEGF receptor-2 (VEGFR2) and serotonin receptors, respectively (FIG. 3A) were assessed. 5 min prior to the infusion of Evans blue dye, shaved 5-to 7-week old mice were injected (10 μl) intradermally with VEGF₁₂₀ (R&D Systems) VEGF₁₆₄ (Chemicon; histamine, Calbiochem) serotonin (Sigma) TIMP-1 (Oncogene Research Products, San Diego Calif.) and the appearance of a blue spot monitored for 30 minutes at which time mice were euthanized, cardiac perfused, photographed and the area of skin surrounding the site of injection excised (˜5 mm²), photographed and Evans blue dye extracted as above. Whereas injection of increasing concentrations of either form of VEGF or serotonin in control mice lead to significant leakage of Evans blue dye into stroma, the response was significantly inhibited in Collα1(I)^{r/r} mice exposed to VEGFA₁₂₀, VEGFA₁₆₄ and serotonin at all concentrations tested (FIG. 3A).

Following intradermal injection of VEGF₁₆₄ (10 ng), tissue lysates were subjected to immunoprecipitation with anti-VEGFR2 antibodies, followed by SDS-PAGE electrophoreses of immune complexes and western blot analysis with anti phospho-tyrosine and anti-VEGFR2 antibodies (FIG. 3B). Tissue pieces (5 mm²) from cardiac-perfused animals previously injected i.d. with 10 μl of either 10 ng VEGF₁₆₄ or 0.1% BSA in PBS were pulverized in liquid N₂ followed by lysis in ice-cold buffer containing 20 mM Tris, pH 7.6, 150 mM NaCl, 1 mM EDTA, 50 mM NaF, 1% triton X-100, 0.5% Na-deoxycholate, 0.1% SDS, 2 mM Na₂VO₄, 10 μg/ml aprotinin, 1 mM phenylmethylsulfonylfluoride and centrifuged at 10,000 rpm for 30 min at 4° C. The supernatants were recentrifuged at 10,000 rpm for 30 min at 4° C. Lysates were then incubated in a slurry of heparin-Sepharose CL-6B (Pharmacia, Peapack, N.J.) and incubated overnight rocking at 4° C., centrifugation and equilibrated to 150 mM NaCl. Protein was dialyzed against PBS and quantified using the BioRad protein assay system (BioRad, Hercules, Calif.). Before immunoprecipitation, BSA was added to the precleared lysates to 0.5%. Equal amounts of protein (1 mg) from lysates was used for immunopreciptations and Western blotting. Incubation of tissue lysate with goat anti-Flk-1 (Santa Cruz Biotechnology, Santa Cruz, Calif.) followed by protein-G sepharose beads was performed for 2 hrs at 4° C. Immunoprecipitates were washed three times with 20 mM Tris (ph 7.6), 150 mM NaCl, 0.1% Triton X-100 and bound proteins were eluted by boiling in 1×SDS-PAGE sample buffer for 5 min, followed by electrophoresis on 10% SDS-PAGE under reducing condition. The resolved proteins were transferred to a nitrocellulose membrane (BA-S85, Schleicher & Schuell, Germany). Anti-phoshoptyrosine PY-20 (Upstate Biotechnology, Lake placid, N.Y.) and anti-Flk-1 (Santa Cruz Biotechnology) antibodies were used on Western blots. Immunodetection was performed by incubation with specific peroxidase-conjugated secondary antibodies followed by enhanced chemiluminescence (ECL, Amersham International plc., Buckinghamshire, UK).

Following exposure to VEGF, activation of VEGFR2 on endothelial cells in control and Collα1(I)^{r/r} mice was suggested by similarly increased levels of phosphorylation of VEGFR2 (FIG. 3B). These data revealed that activation of VEGFR2, as evidenced by its phosphorylation following VEGF binding, occurred to a similar degree in control and Collα1(I)^{r/r} mice; thus, suggesting that VEGF was not sequestered by mutant collagen per se, and that the attenuated vascular permeability response in Collα1(I)^{r/r} mice was not due to sequestration of VP-inducing agents.

### EXAMPLE 4

### Vascular Perfusions and Fluorescent Angiography

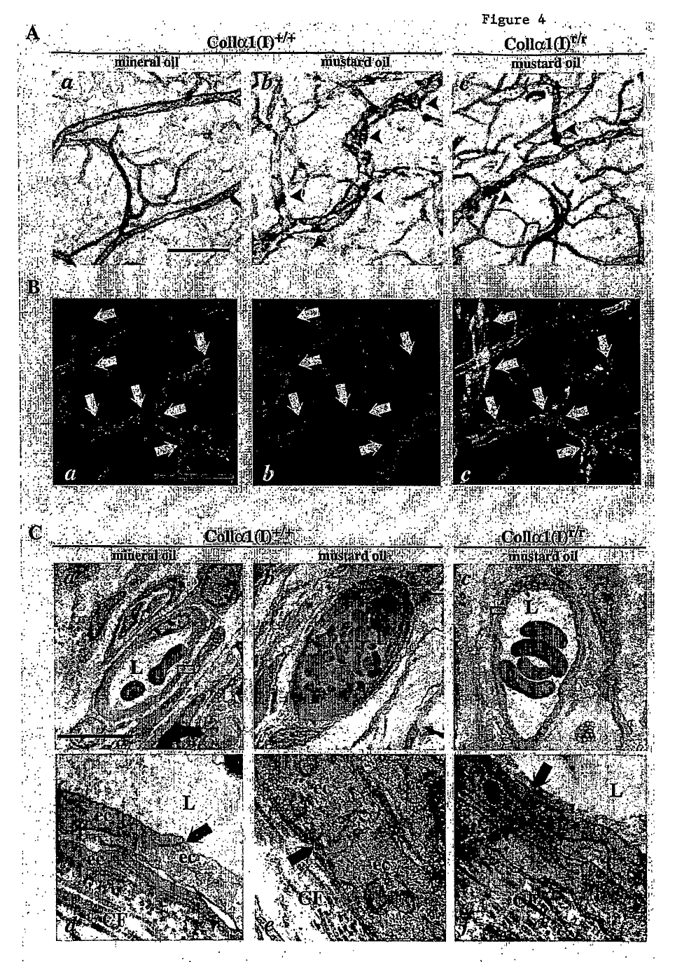

The Collα1(I)^{r/r} mice and control mice were injected (i.v.) with fluorescein Lycopersicon esculentum lectin (100 μl, 2 mg/ml) and Rhodamine Ricinus communis agglutinin I (50 μl, 5 mg/ml), a lectin that specifically binds capillary luminal openings and exposed regions of basement membrane at sites of interendothelial gaps (Hashizume (1998) Br J Dermatol 139: 1020-1025) followed by fluorescent angiography and confocal visualization (FIG. 4A-B) Isoflurane-anesthetized mice were injected with 20 ml of 5 mg/ml labeled-Lycopersicon esculentum (tomato) lectin (Vector Laboratories, Burlingame, Calif.), or 20 ml of 10 mg/ml labeled-Ricinus communis (castor bean) lectin (Vector Laboratories) into the femoral vein. Two minutes after lectin injection, mice were perfused with fixative (1% paraformaldehyde plus 0.5% glutaraldehyde in phosphate-buffered saline, pH 7.4, at 37° C.) via the ascending aorta for 2 min to fix the vasculature and wash out non-adherent leucocytes. All the analyses were carried out on groups of at least three mice. Confocal analysis of whole mount ears in this experiment revealed decreased appearance of sites of vascular leakage as revealed by less Rhodamine Ricinus communis agglutinin I staining in MO-treated Collα1(I)^{r/r} skin as compared to MO-treated control skin (compare FIG. 3A panel b with 3A panel c). Moreover, the appearance of leakage sites seemed to be concentrated along certain regions of the vasculature. Sites of vascular leakage following MO treatment in Ricinus communis Agglutin I-injected control mice in combination with histochemical detection of alpha-smooth muscle actin (αSMA) in whole mount tissues (FIG. 4B) were analyzed. This analysis revealed specific regions of the vasculature, as demonstrated by the presence, absence or phenotype of αSMA-positive cells. Thus, vascular leakage (as indicated by presence of ricin binding) in response to MO occurred prominently in regions of vasculature either devoid of αSMA-positive capillary support cells or in regions where the morphology of αSMA-positive cells was consistent with the morphology of pericytes present on post-capillary venules (FIG. 4B) (Benjamin (2000) Cancer Metastasis Rev 19, 75-81).

Control and Collα1(I)^{r/r} skin following MO (or vehicle) treatment was analyzed on an ultrastructural level (FIG. 4C). Briefly, ear skin pieces were collected following cardiac perfusion, thinly sliced (˜1 mm thick) and placed in Karnovsky's fixative (1% para-formaldehyde, 3% glutaraldehyde, 0.1 M sodium cacodylate buffer, pH 7.4) at room temperature for 30 minutes before storage at 4° C. Fixed tissue were then rinsed in water, post-fixed in reduced OsO₄ (2% OsO₄ in 1.5% potassium ferrocyanide; Sigma Chemical), stained en bloc with uranyl acetate before dehydration in 100% ethanol, cleared in propyline oxide, and embedded in Eponate 12 (Ted Pella Co., Redding, Calif.). Thick section were cut and stained with toluidine blue, examined under light microscope to select areas for subsequent thin sectioning. Thin sections were cut with a Leica ultracut E microtome (Bannockburn, Ill.), stained with uranyl acetate and Reynold's Lead to enhance contrast and examined with a Philips Tecnai 10 electron microscope (Eidhoven, The Netherlands).

Presence of hyperpermeable fenestrae were not observed in control or Collα1(I)^{r/r} tissue following exposure to vehicle or MO (data not shown). In contrast, following exposure of control mice to MO, endothelial cell opening were readily observed in capillaries devoid of perivascular support cells (FIG. 4C panel b and e). In Collα1(I)^{r/r} skin, presence of endothelial cell opening could not be documented in similar vascular regions following extensive examination (FIG. 4C, panels c and f). Therefore, mutant collagen in the vascular stroma renders vascular cells less susceptible to vasodilation following stimulation resulting in restricted opening in or between endothelial cells resulting in diminished vascular leakage, thus reducing plasma protein extravasation from vascular lumens into perivascular stroma.

### EXAMPLE 5

### Type I Collagen Accumulation Regulates Vascular Hyperpermeability

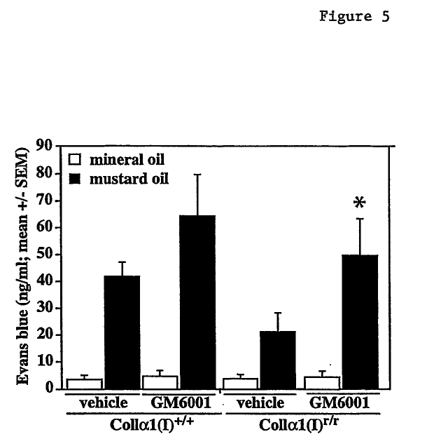

Vascular permeability (VP) responses in control and Collα1(I)^{r/r} mice treated with a broad spectrum synthetic metalloproteinase inhibitor (MPI), e.g., GM6001 were examined. GM6001 (N-[(2R)-2(hydroxyamidocarbonylmethyl)-4-methylpantanoyl]-L-tryptophan ethylamide), a broad, class-specific metalloproteinase inhibitor (Chemicon, Temecula Calif.), was administered daily i.p. at 100 mg/kg body weight as a 20 mg/ml slurry in 4% carboxymethylcellulose in 0.9% PBS daily for 3-days. Controls were treated with a daily injection of 4% carboxymethylcellulose in PBS. The animals were then subject to cutaneous challenge with MO and qualitative and quantitative assessment of Evans blue dye leakage into vascular stroma (FIG. 5A). MO-exposure to GM6001 treated control mice resulted in a characteristic increase of Evans Blue dye leakage into vascular stroma, higher than that observed in MO-treated control mice receiving vehicle alone (FIG. 5A). Similarly, MO-treatment of GM6001 treated Collα1(I)^{r/r} mice resulted in increase of Evans blue leakage, significantly above vehicle-treated Collα1(I)^{r/r} mice (FIG. 5A); thus, GM6001 treatment restored a characteristic VP response to Collα1(I)^{r/r} mice and rendered control mice somewhat hyperpermeable and more susceptible to vascular leakage following stimulation.

### EXAMPLE 6

### Increased MMP2 Activity in Collα1(I)^{r/r} Mice

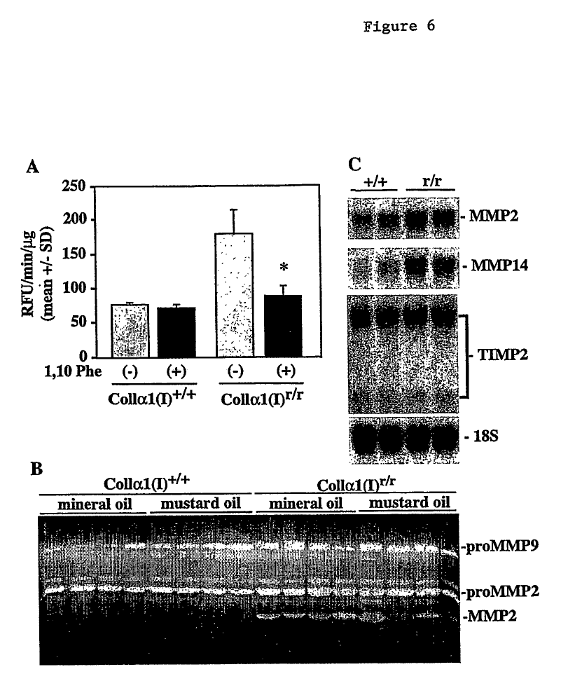

The substrate conversion assay with quenched fluorescently-labeled gelatin as a substrate and tissue lysates from control and Collα1(I)^{r/r} skin (FIG. 6A) was used to assess the proteolytic activity of Collα1(I)^{r/r} mice toward gelatin, a common matrix metalloproteinase (MMP) substrate. Tissue pieces from 5-8 week old mice were pulverized in liquid N₂ and solubilized in 500 μl buffer (0.25 M sucrose, 5 mM Tris, pH=7.5, Protease Inhibitor cocktail mix without EDTA (Roche), 0.25 mg/ml Pefablock (Roche), 0.01 mg/ml Aprotinin (Sigma) using a 2 ml tissue grinder (Fisher) and centrifuged at 4° C. 800×g for 15 min. Supernatants were again centrifuged for 1 hr at 100,000×g at 4° C. Supernatants were stored at −80° C., pellets were resuspended in 100 μl solubilization buffer, homogenized by sonication at 4° C., and stored at −80° C. Protein concentration was determined with the BioRad DC Protein assay reagent according to manufacturers instructions (BioRad). Prior to assay, lysate buffers were exchanged using Micro Bio-spin chromatography columns (Bio-gelP-6; Biorad) to 10 mM Tris pH 7.5 according to the manufacturers specifications. For assay of gelatinolytic activity in lysates, 50 μg protein from the supernatant fraction was incubated at 37° C. with 400 ng DQ-gelatin (Molecular Probes, Eugene, Oreg.) in reaction buffer (50 mM Tris, pH 7.6, 150 mM NaCl, 5 mM CaCl₂, 0.2 mM NaAzide and 0.05% BrJ35) in a total volume of 200 μl/well (black 96-well plate, Falcon). Reactions were incubated up to 5 hr at 37° C. and fluorescence measured (excitation 485 mn, emission 530 nm) every 3 min on a Microplate Spectrofluorometer (SpectraMax Gemini EM, Molecular Devices, Sunnyvale, Calif.) and quantified using SoftMax Pro 4.1 software. Values shown represent the mean +/− SEM from three tissue pieces. All analyses were performed a minimum of three times and are representative. This analysis revealed a 6-fold higher gelatinolytic activity in Collα1(I)^{r/r} skin (p<0.04, Fishers, 2-tailed) compared to that of control mouse skin, that was completely inhibited by treatment with 1,10 phenanthroline (4 mM), a MMP inhibitor (FIG. 6A).

The skin lysates from control and Collα1(I)^{r/r} mice were examined by gelatin substrate zymography to visualize differences in gelatinolytic enzymes between the two genotypes (FIG. 6B). Tissue samples from 5-8 week old mice were weighed and then homogenized (1:8 weight to volume) in lysis buffer containing 50 mM Tris-HCl (pH 8.0), 150 mM NaCl, 0.1% NP-40, 0.5% deoxycholate, 0.1% SDS. Soluble and insoluble extracts were separated by centrifugation (10,000×g) and subsequently stored at −80° C. Equivalent amounts of soluble extract were analyzed by gelatin zymography on 10% SDS-polyacrylamide gels copolymerized with substrate (1 mg/ml of gelatin) in sample buffer (2% SDS, 50 mM Tris-HCl, 10% Glycerol, 0.1% Bromphenol Blue, pH 6.8). After electrophoresis, gels were washed 3 times for 30 min in 2.5% Triton X-100, 3 times for 15 min in ddH₂O, incubated overnight at 37° C. in 50 mM Tris-HCl, 10 mM CaCl₂ (pH 8.2), and then stained in 0.5% Coomassie Blue and destained in 20% methanol, 10% acetic acid. Negative staining indicates the location of active protease bands. Exposure of proenzymes within tissue extracts to SDS during gel separation procedure leads to activation without proteolytic cleavage. Gelatin substrate zymographic analysis of tissue lysates revealed no change in abundance of proMMP9 or proMMP2, but instead revealed increased presence of the lower molecular weight form of active MMP2 in Collα1(I)^{r/r} skin as compared to control skin, independent of prior exposure to MO (FIG. 6B).

The MMP2 mRNA levels in control and Collα1(I)^{r/r} skin were determined by northern blot analysis (FIG. 6C). Total RNA was extracted from skin pieces with TRIzol reagent™ (Invitrogen) according to the manufacturer's recommendations, by powdering fresh-frozen tissue samples in liquid N₂, homogenizing with a microtube pestle (USA Scientific), shearing by multiple passages through a syringe and 21-gauge needle (Becton Dickinson), followed by chloroform extraction, isopropanol precipitation and ethanol wash. Northern blot analysis was performed using standard methods with 10 μg of total cellular RNA. Probes were generated by random primed labeling of DNA isolated from plasmids using standard methodology. Northern blots were probed at 65° C. overnight, and subsequently washed at 62° C. in 2×SSC containing 1% SDS. Probes used for hybridization were: a fragment of mMMP2, a fragment of mMMP14, a fragment of mTIMP2 (Shimizu.-S., et al (1992) Gene 114: 291-292), a fragment of mTGFβ1 and a fragment for 18S RNA as control. Hybridized filters were subjected to analysis in a Phosphoimager. This analysis revealed an −1.5-fold increase in MMP2 mRNA as compared to a control MRNA (FIG. 6C, top panel). In addition, since MMP14 and TIMP2 have been implicated in regulating activation of proMMP2 on the plasma membrane, we assessed MMP14 and TIMP2 mRNA levels and also found MMP-14 mRNA levels to be modestly increased 2.8-fold above that in control mice, whereas no difference in TIMP-2 MRNA between the two genotypes was found. Thus, while a modest increase in MMP2 and MMP-14 mRNA was found in Collα1(I)^{r/r} mice compared to controls, the 6-fold higher activity in gelatinoloytic activity in Collα1(I)^{r/r} skin lysates suggests that increased presence of the low molecular weight form of MMP2 results from post-translational activation of latent proMMP2.

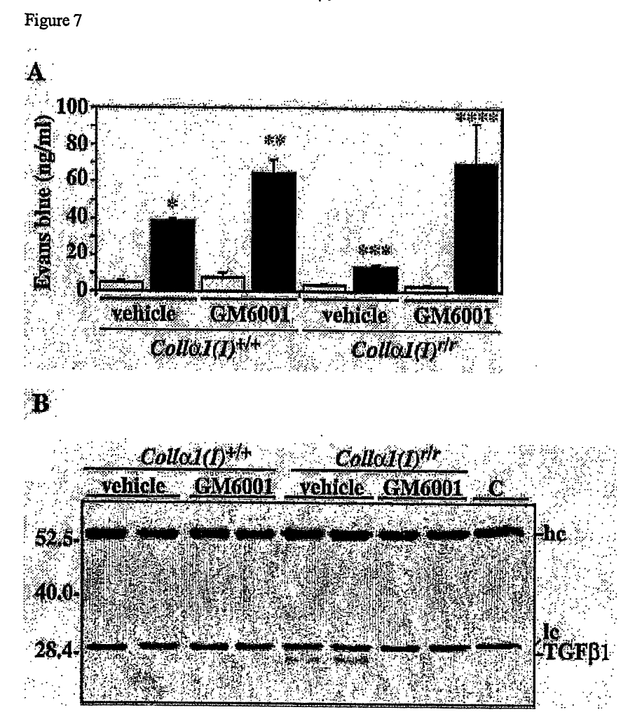

Several mechanisms for activation of latent TGFβ complexes have been proposed, including cleavage of LAP by serine and metallo-proteases, and interaction with thrombospondin-1, αvβ6 integrins, reactive oxygen species (ROS) and low pH (Annes, J. P., Munger, J. S. & Rifkin, D. B. (2003) J Cell Sci 116, 217-224). Stabilized and/or highly cross-linked forms of type I collagen fibrils in vitro induce MMP mRNA, e.g., MT1-MMP/MMP14, as well as activation of latent MMP activity, e.g., MMP1, MMP2 and MMP14, the latter two proteases also being implicated in activating latent TGβ. To determine if this increased MP activity in Collα1(I)^{r/r} tissue was functionally relevant in regulating their abnormal vascular responses, Collα1(I)^{+/+} and Collα1(I)^{r/r} mice were treated with a broad-spectrum synthetic metalloproteinase inhibitor (MPI), e.g., CM6001, followed by challenge with MO and assessment of EB leakage. Administration of GM6001 restored the appropriate acute vascular responses in Collα1(I)^{r/r} mice as assessed by EB leakage and was significantly higher than MO-stimulated vehicle-treated Collα1(I)^{r/r} mice (p=0.0388, unpaired t test; FIG. 7A). Surprisingly, administration of GM6001 rendered control mice even more susceptible to MO-induced EB leakage compared to controls (p=0.0247, unpaired t test; FIG. 7A). In addition, use of the MPI in Collα1(I)^{r/r} mice markedly decreased levels of the ˜25 kDa dimeric mature form of TGFβ₁ (FIG. 7B). Taken together, these data suggest that “stabilization” of type I collagen fibrils in the perivascular stroma from Collα1(I)^{r/r} mice indirectly results in MMP-mediated proteolytic activation of latent TGFβ₁.

### EXAMPLE 7

### TGFβ Blocks Induction of Vascular Permeability

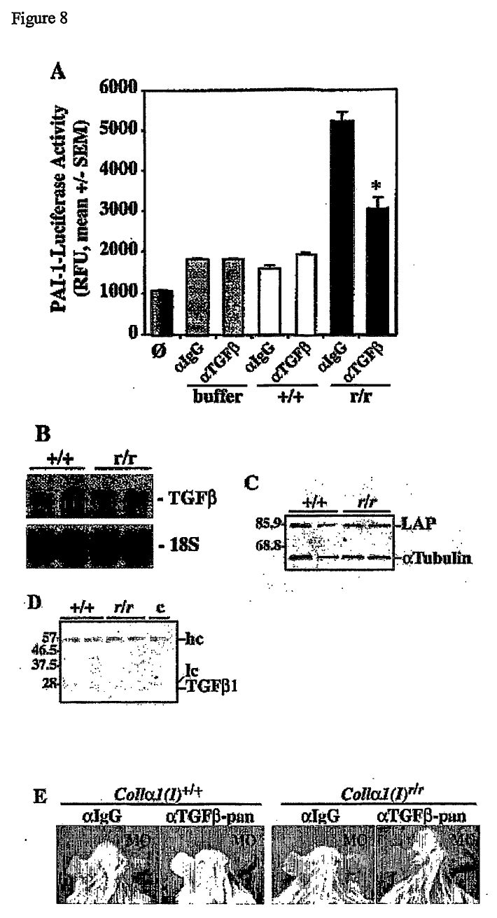

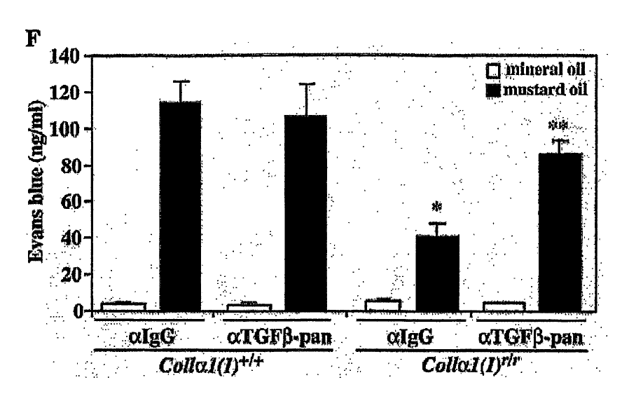

The TGFβ activity in tissue lysates from Collα1(I)^{r/r} and control mice variably treated with MO (FIG. 8A) was examined utilizing a bioassay for TGFβ activity (Abe (1994) Anal Biochem 216: 276-284). Mink lung epithelial cells (MLECs) stably-transfected with a construct containing a truncated PAI-1 promoter element fused to the firefly luciferase reported gene (PAI-1-luciferase construct) were used as described (Abe (1994) above). Cells were maintained in high glucose (4500 mg/liter) Dulbecco's Modified Eagle's Medium (DMEM) containng 10% fetal calf serum, 2 mM L-glutamine, 1 mM sodium pyruvate and 200 μg/ml Geneticin (G418-sulfate). Prior to assay, cells were grown for 24 hr in “serum-free’ medium supplemented with 0.1% bovine serum albumin (Gibco), trypsinized, washed several times in serum-free medium and plated at 1.6×10⁵ cells/ml, 400 μl per well, into 24-well tissue culture plates (Becton Dickinson) and allowed to attach for 3 h at 37° C. The medium was then replaced with activated standards or samples in DMEM/BSA in triplicate. Tissue samples were prepared from skin pieces removed from animals previously perfused with a potassium-free PBS infusion in the right ventricle of the heart to clear vasculature of blood. Tissue lysates were then made by pulverizing tissue in liquid N₂ and stirring powder at 37° C. for 1 h in 50 mM Tris-Hcl (pH 7.5), 75 mM NaCl, 10 mM EDTA containing a protease inhibitor cocktail (Rocke) in a sterile spinnerflask, followed by centrifugation at 4° C. for 15 min at 10,000 g and stored at −80° C. with 2.5 μl of 0.2 M phenylmethylsulfonyl fluoride (Sigma) and 0.05 units of aprotinin (Sigma) per milliliter of tissue extract. 100 μg of tissue lysates were added to MLEC and resulting luciferase activities were measured 16 hr later by the Luciferase Assay System (Promega Corp, Madison, Wis.) according to the manufacturer's instructions. Recombinant human transforming growth factor-β1 and neutralizing antibodies directed against TGF-β were from R & D Systems. Mink lung epithelial cells stabley transfected with a plasminogen activator type I PAI-1) promoter regulating a luciferase reporter gene were incubated with increasing amounts of tissue lysate from Collα1(I)^{r/r} versus control mice variably treated with MO (FIG. 8B). Tissue lysates from Collα1(I)^{r/r} mice consistently yielded higher luciferase activity in cells as compared to lysates from control mice -activity that was specifically blocked by incubation of lysates with a neutralizing antibody to all three isoforms of TGFβ (FIG. 8A). The total TGFβ₁ measured in skin lystaes using an ELISA was found to be ˜2-fold higher in Collα1(I)^{r/r} mice compared with controls (p=0.02, unpaired t test). These differences were not accounted for by increased expression of TGFβ₁ since there was no difference in levels of MRNA (FIG. 8B) or in the levels of TGFβ₁ latency-associated peptide (LAP: FIG. 8C). Levels of the ˜25 kDa dimeric, mature TGFβ₁, however, were clearly increased in tissue from Collα1(I)^{r/r} compared to Collα1(I)^{+/+} mice (FIG. 8D). Thus, the increased levels of TGFβ₁ in Collα1(I)^{r/r} mice reflected increased local activation of latent TGFβ₁ rather than increased transcription, synthesis or secretion.

Neutralizing antibodies to TGFβ₁ were administered to control and Collα1(I)^{r/r} mice, prior to MO challenge for 6-days prior to cutaneous challenge with MO (FIGS. 8E and F). Neutralization of all TGFβ isoforms in Collα1(I)^{r/r} mice resulted in complete restoration of EB leakage following MO-stimulation to a level similar to that in Collα1(I)^{+/+} mice (FIGS. 8E and F), suggesting that local activation of TGFβ in Collα1(I)^{r/r} mice restricts vascular activation and leakage following acute stimulation. Therefore, TGFβ bioavailabilty is regulated post-translationally by a type I collagen and MMP-sensitive pathway, and together act as critical extracellular sensors regulating rapid induction of vascular permeability and plasma protein extravasation in response to acute trauma.

While the invention has been particularly shown and described with reference to a preferred embodiment and various alternate embodiments, it will be understood by persons skilled in the relevant art that various changes in form and details can be made therein without departing from the spirit and scope of the invention. All printed patents and publications referred to in this application are hereby incorporated herein in their entirety by this reference.

We claim:

1. A method for modulating vascular permeability in a subject, the method comprising administering to a subject in need of treatment an effective amount of a therapeutic agent to modulate the level and/or activity of TGF-β wherein the therapeutic agent modulates the vascular permeability.

2. The method of claim 1, wherein the therapeutic agent inhibits the production or bioavailability of TGF-β or the expression of TGF-β.

3. The method of claim 2, wherein the therapeutic agent inhibits the bioavailablity of TGF-β.

4. The method of claim 3, wherein the therapeutic agent is an antisense oligonucleotide.

5. The method of claim 1, wherein the therapeutic agent stimulates the production or bioavailability of TGF-β or the expression of TGF-β.

6. The method of claim 5, wherein the therapeutic agent increases the bioavailablity of TGF-β.

7. The method of claim 1, wherein the therapeutic agent is an antagonist.

8. The method of claim 7, wherein the antagonist is an oligonucleotide.

9. The method of claim 7, wherein the antagonist is a small molecule.

10. The method of claim 9, wherein the antagonist is selected from the group consisting of SB-431542, NPC-30345, and LY-364947.

11. The method of claim 10, wherein the antagonist is SB-431542.

12. The method of claim 10, wherein the antagonist is NPC-30345.

13. The method of claim 10, wherein the antagonist is LY-364947.

14. The method of claim 7, wherein the therapeutic agent is a monoclonal antibody.

15. The method of claim 14, wherein the monoclonal antibody is selected from the group consisting of ID11 and 2G7.

16. The method of claim 15, wherein the monoclonal antibody is ID11.

17. The method of claim 15, wherein the monoclonal antibody is 2G7.

18. The method of claim 14, wherein the monoclonal antibody is a humanized monoclonal antibody selected from the group consisting of CAT-152 and CAT-192.

19. The method of claim 18, wherein the humanized monoclonal antibody is CAT-152.

20. The method of claim 18, wherein the humanized monoclonal antibody is CAT-192.

21. The method of claim 7, wherein the therapeutic agent is a polyclonal antibody.

22. The method of claim 1, wherein the therapeutic agent is an agonist.

23. The method of claim 22, wherein the agonist is an oligonucleotide.

24. The method of claim 22, wherein the agonist is a small molecule.

25. The method of claim 24, wherein the molecule is selected from the group consisting of tamoxifen, aspirin, aspirinate and salts thereof.

26. The method of claim 25, wherein the molecule is tamoxifen, and salts thereof.

27. The method of claim 25, wherein the molecule is aspirinate, and salts thereof.

28. The method of claim 1, wherein the therapeutic agent is an anti-fibrotic agent reducing collagen synthesis.

29. The method of claim 28, wherein the therapeutic agent is Halofuginone.

30. The method of claim 1, wherein the therapeutic agent is an anti-fibrotic agent reducing collagen crosslinking.

31. The method of claim 30, wherein the anti-fibrotic agent is a transglutaminase or a reducing sugar.

32. The method of claim 31, wherein the agent is a reducing sugar.

33. The method of claim 31, wherein the anti-fibrotic agent is an enzyme selected from the group consisting of horseradish peroxidase, soybean peroxidase, and peroxidase from Arthomyces ramosus.

34. The method of claim 33, wherein the enzyme is horseradish peroxidase.

35. The method of claim 33, wherein the enzyme is soybean peroxidase.

36. The method of claim 33, wherein the enzyme is peroxidase from Arthomyces ramosus.

37. The method of claim 1, wherein the therapeutic agent is a protease inhibitor.

38. The method of claim 37, wherein the protease inhibitor is a serine protease inhibitor or a urokinase inhibitor.

39. The method of claim 38, wherein the protease inhibitor is serine protease inhibitor.

40. The method of claim 38, wherein the protease inhibitor is a urokinase inhibitor.

41. The method of claim 1, wherein vascular permeability is associated with wound healing.

42. The method of claim 1, wherein vascular permeability is associated with disease states selected from the group consisting of diabetic retinopathy, psoriasis, cancer, rheumatoid arthritis, atheroma, Kaposi's sarcoma and haemangioma.

43. The method of claim 43, wherein the disease state is cancer, and the cancer is breast cancer or prostate cancer.

44. The method of claim 42, wherein the disease state is rheumatoid arthritis.
